(19) United States
(12) Patent Application Publication
(10) Pub. No.: US 2023/0071314 A1
(43) Pub. Date: Mar. 9, 2023

(54) POLYMERIC COMPOSITIONS FOR SUSTAINED DELIVERY OF VMAT2 INHIBITORS

(71) Applicant: Foresee Pharmaceuticals Co., Ltd., Taipei (TW)

(72) Inventors: Yuhua LI, Newark, DE (US); Shih-Fan JANG, New Taipei City (TW); Wen-Yen HUANG, New Taipei City (TW); Chia-Ting HUANG, New Taipei City (TW)

(21) Appl. No.: 17/889,009

(22) Filed: Aug. 16, 2022

Related U.S. Application Data
(60) Provisional application No. 63/233,659, filed on Aug. 16, 2021.

(51) Int. Cl. A61K 9/00; A61K 47/32; A61K 31/4375

(52) U.S. Cl. CPC A61K 9/0024; A61K 47/32; A61K 31/4375

(57) ABSTRACT

The present application relates to a sustained release delivery composition of a vesicular monoamine transporter type 2 (VMAT2) inhibitor, a deuterated derivative thereof, a pharmaceutically acceptable salt thereof, an active metabolite thereof, or a prodrug thereof for treatment of hyperkinetic movement disorders including, but not limited to, tardive dyskinesia (TD), Huntington's disease (HD) chorea, tremors, dystonia, chorea, tics, myoclonus, stereotypies, restless legs syndrome, and various other disorders with abnormal involuntary movements. The method of making or using the composition is also disclosed.

### CROSS REFERENCE TO RELATED APPLICATION

This application claims the benefit of U.S. Provisional Patent Application No. 63/233,659 filed on Aug. 16, 2021, the disclosure of which is incorporated herein by reference in its entirety.

### BACKGROUND OF THE APPLICATION

### 1. Field of the Application

The present application provides for a biodegradable polymer depot composition which is stable and effective as a sustained release delivery system for the reversible human vesicular monoamine transporter type 2 (VMAT2) inhibitors. The composition of the present application comprises a) a VMAT2 inhibitor, including but not limited to, (3R,11bR)-tetrabenazine [(+)-TBZ, (3R,11bR)-1,3,4,6,7,11b-hexahydro-9,10-dimethoxy-3-(2-methylpropyl)-2H-benzo[a]quinolizin-2-one], (2R,3R,11bR)-dihydrotetrabenazine [(+)-(α)-DHTBZ, (2R,3R,11bR)-3-isobutyl-9,10-dimethoxy-1,3,4,6,7,11b-hexahydro-2H-pyrido[2,1-a]isoquinolin-2-ol)], (2S,3R,11bR)-dihydrotetrabenazine [(+)-(β)-DHTBZ, (2S,3R,11bR)-3-isobutyl-9,10-dimethoxy-1,3,4,6,7,11b-hexahydro-2H-pyrido[2,1-a]isoquinolin-2-ol)], a deuterated derivative thereof, a pharmaceutically acceptable salt thereof, an active metabolite thereof, or a prodrug thereof; b) one or more biodegradable, biocompatible, polymeric carriers; c) one or more pharmaceutically acceptable and biocompatible solvents; and d) one or more optional pharmaceutically acceptable excipients allowing the achievement of optimizing drug delivery. The present application also provides a method of manufacturing and the use in treating hyperkinetic diseases and disorders, such as tardive dyskinesia, by administration of such composition to human or a warm-blooded animal in need thereof.

### 2. Description of the Related Art

Tardive dyskinesia (TD) is a hyperkinetic movement disorder resulting in involuntary, repetitive body movements which are not related to other disorders provoking the aforementioned involuntary movements, for example, Parkinson's disease or tic disorders. Instead, TD is a neurological disorder most commonly caused by long-term use of dopamine blocking agents such as antipsychotic drugs (also known as neuroleptics or dopamine receptor antagonists). First generation neuroleptics (typical neuroleptics, for example haloperidol and chlorpromazine) are very likely to cause TD; while newer neuroleptics (atypical neuroleptics, for example aripiprazole and paliperidone), on the other hand, can do the same but to a lesser extent.

Prior arts suggest continuous exposure to neuroleptics can cause upregulation/supersensitiveness of dopamine receptor, which then induces hyperkinetic movement disorder. Vesicular monoamine transporter-2 (VMAT2) is a membrane protein that transports monoamine, such as dopamine, from presynaptic into synaptic vesicles. Many hyperkinetic movement disorders, namely TD, Tourette syndrome, and Huntington's disease can be reduced through depleting presynaptic dopamine by VMAT2 inhibitors. Tetrabenazine (TBZ, brand name XENAZINE®), known as cis-rac-1,3,4,6,7,11b-hexahydro-9,10-dimethoxy-3-(2-methylpropyI)-2H-benzo[a]quinolizin-2-one, is a potent and reversible inhibitor for human VMAT2 Ki˜10 0nM, XENAZINE® Drug Approval Package, NDA 021894. However, while TBZ is orally administered as racemic mixtures, it is rapidly metabolized (majorly in the liver by carbonyl reductase) into four stereoisomeric metabolites: R,R,R-DHTBZ ((+)-α), S,R,R-DHTBZ ((+)-β), S,S,S-DHTBZ ((−)-α), and R,S,S-DHTBZ ((−)-β) (DHTBZ, dihydrotetrabenazine, 9,10-dimethoxy-3-(2-methylpropyl)-2,3,4,6,7,11b-hexahydro-1H-benzo[a]quinolizin-2-ol) (Skor H. et el., Drugs R D. 2017 September; 17(3):449-459). However, each metabolite shows varied affinity to rat VMAT2: Ki is 4.2, 9.7, 250, and 690 nM, respectively corresponding to R,R,R-DHTBZ ((+)-α), S,R,R-DHTBZ ((+)-β), S,S,S-DHTBZ ((−)-α), and R,S,S-DHTBZ ((−)-β) (Grigoriadis et al., Journal of Pharmacology and Experimental Therapeutics June 2017, 361 (3) 454-461). In addition, S,S,S-DHTBZ ((−)-α) and R,S,S-DHTBZ ((−)-β) have high off-target binding affinity to dopamine D2 and serotonin 5-HT₇ receptors (180/71 nM and 53/5.9 nM for ((−)-α) and ((−)-β), respectively), which results in severe side effects of TBZ administration (i.e. insomnia, tremor, rigid muscle, problems with balance etc.) (Harriott et al., Progress in Medicinal Chemistry Volume 57, 2018, Pages 87-111). Moreover, due to the variable CYP 2D6-mediated metabolism of TBZ, the maintenance dose of TBZ varies from one individual to another, therefore, CYP 2D6 inducers or inhibitors should also be avoided for subjects taking TBZ. What's even more significant and potentially inconvenient is that metabolism variation between patients makes dose titration unavoidable for conventionally available TBZ medications. Furthermore, the side effects related to TBZ such as sedation, depression, akathisia and Parkinsonism and therapeutic variability have impeded its application potential.

In 2017, two new medications were approved to treat TD: Valbenazine (VBZ) (INGREZZA®, Neurocrine Biosciences, Inc., single 40 mg or 80 mg capsule per day) and deutetrabenazine (AUSTEDO®, Teva, 6 mg, 9 mg, or 12 mg tablet, twice daily). Unlike TBZ, deutetrabenazine and VBZ have pharmacokinetic advantages which enable less frequent dosing for better tolerability. VBZ, L-Valine, (2R,3R,11bR)-1,3,4,6,7,11b-hexahydro-9,10-dimethoxy-3-(2-methylpropyl)-2H-benzo[α]quinolizin-2-yl ester, is an ester of (+)-(α)-DHTBZ with the amino acid L-valine. By solely introducing (+)-(α)-DHTBZ without the presence of the other side effect inducing stereoisomeric metabolites, such as (−)-(α)-DHTBZ and (−)-(β)-DHTBZ, VBZ is considered much more tolerable and safer than TBZ. On the other hand, in the case of AUSTEDO®, deuterated derivative of TBZ increases the half-life of deutetrabenazine which benefits for the reduced dosing frequency.

Although the success of INGREZZA® and AUSTEDO® improve TD treatment in oral dosage forms, both products still require daily dosing, which is not ideal from improving patient adherence point of view. Poor compliance remains the most critical challenge in the treatment of any chronic illness. Schizophrenia, for example, is often associated with cognitive dysfunction, lack of motivation, depression, and demoralization. While the introduction of antipsychotics can be backdated to the 1950s, poor adherence to oral dosage forms has always been a crucial issue. Relapse is a continual risk in schizophrenia patients and represents one of the major public health problems associated with such illness. The use of long-acting injectables (LAIs) alleviates the burden of frequent administration which helps avoid poor/partial adherence. While there are already many LAI medicines launched on the market for treating bacterial infections, pain management, prostate cancer, diabetes, and certain schizophrenia employing various formulative technologies, such as ATRIGEL®, SABER® and FluidCrystal® etc., a successful LAI drug product on hyperkinetic movement disorders hasn't been developed yet. While patients taking antipsychotic drugs receive the benefits from LAI antipsychotics, they still have to take daily pills (INGREZZA® or AUSTEDO®) once involuntary movement is developed. This surely is still troublesome from patient adherence point of view. Therefore, there is definitely unmet medical need for a stable and safer LAI medication for the treatment of involuntary movement disorder with significantly reduced dosing frequency and improved patient compliance.

### SUMMARY OF THE APPLICATION

The present application provides polymer depot compositions comprising of a) a VMAT2 inhibitor, including but not limited to, tetrabenazine (TBZ), (3R,11bR)-tetrabenazine [(+)-TBZ, (3R,11bR)-1,3,4,6,7,11b-hexahydro-9,10-dimethoxy-3-(2-methylpropyl)-2H-benzo[a]quinolizin-2-one], (2R,3R,11bR)-dihydrotetrabenazine [(+)-(α)-DHTBZ, (2R,3R,11bR)-3-isobutyl-9,10-dimethoxy-1,3,4,6,7,11b-hexahydro-2H-pyrido[2,1-a]isoquinolin-2-ol)], (2S,3R,11bR)-dihydrotetrabenazine [(+)-(β)-DHTBZ, (2S,3R,11bR)-3-isobutyl-9,10-dimethoxy-1,3,4,6,7,11b-hexahydro-2H-pyrido[2,1-a]isoquinolin-2-ol)], a deuterated derivative thereof, a pharmaceutically acceptable salt thereof, an active metabolite thereof, or a prodrug thereof; b) one or more biodegradable, biocompatible, polymeric carriers; c) one or more pharmaceutically acceptable and biocompatible solvents; and d) one or more optional pharmaceutically acceptable excipients allowing the achievement of optimal drug delivery for intended uses.

The present application relates to a long-acting injectable delivery system of (+)-TBZ, (+)-(α)-DHTBZ, (+)-(β)-DHTBZ, deuterated derivative thereof, a pharmaceutically acceptable salt thereof, an active metabolite thereof, or a prodrug thereof, which have high VMAT2 receptor binding affinity (<10 nM), but low off-target binding to such as dopamine, serotonin, and adrenergic receptors (>1000 nM).

Appropriately, the present application provides a stable, biodegradable composition that is effective as an in situ forming depot allowing prolonged, controlled release of (+)-TBZ, (+)-(α)-DHTBZ, (+)-(β)-DHTBZ, a deuterated derivative thereof, a pharmaceutically acceptable salt thereof, an active metabolite thereof, or a prodrug thereof. The present polymer depot compositions can be a viscous fluid, a solution, a gel, an emulsion, a suspension, or a semisolid dispersion that is preserved in a readily pre-filled syringe for subcutaneous or intramuscular injection. The polymer depot compositions can also be stabilized and preserved in two separated syringes, i.e., one syringe contains the active pharmaceutical ingredient and the other syringe contains the delivery vehicle. After adequate mixing of the two syringes, the final mixture can be a viscous fluid, a solution, a gel, an emulsion, a suspension, or a semisolid dispersion for subcutaneous or intramuscular injection.

Specifically, the present application is capable of forming a sustained release implant/depot upon administration to a living subject at the injection site. Preferably, the inventive compositions are competent for maintaining long-term plasma concentration of (+)-TBZ , (+)-(α)-DHTBZ, (+)-(β)-DHTBZ and active metabolites above therapeutic level preferably for 1 to 2 weeks, more preferably for 2 to 4 weeks, and most preferably for 1 to 3 months with minimum variation in plasma concentration and narrow peak-to-trough (P/T) ratio, which can limit potential off-target effect (resulted from the (−) stereoisomers of TBZ and DHTBZ) so as to ultimately provide an improved safety profile to solve the unmet medical need of currently available drug products on the market.

### BRIEF DESCRIPTION OF THE FIGURES

FIG. 1 and FIG. 2 respectively show the “Resomer® Select naming” published by Evonik in the published “RESOMER® product brochure.”

FIG. 3 shows molecular weight information of Pskitr1L ReadyCal-Kit.

FIG. 4A shows sustained release of (+)-TBZ polymer depot compositions, and FIG. 4B shows sustained release of (+)-(α)-DHTBZ polymer depot compositions. As described in Table 1, particle D(50) values for small (S) and large (L) were 10-35 μm and 100-130 μm, respectively.

FIG. 5A and FIG. 5B respectively show effect of polymer/NMP ratio on sustained release of (+)-TBZ polymer depot compositions and (+)-(α)-DHTBZ polymer depot compositions. Particle D(50) values for small (S) and large (L) were 10-35 μm and 100-130 μm, respectively.

FIG. 6 shows effect of hydrophobic solvent additive on sustained release of (+)-(α)-DHTBZ polymer depot compositions. Particle D(50) value for large (L) was 100-130 μm.

FIG. 7A, FIG. 7B, and FIG. 7C respectively show effect of drug loading % on sustained release of (+)-TBZ polymer depot compositions (FIGS. 7A and 7B) and (+)-(α)-DHTBZ polymer depot compositions (FIG. 7C). Particle D(50) values for small (S), medium (M), and large (L) were 10-35 μm, 50-80 μm, and 100-130 μm, respectively.

FIG. 8A, FIG. 8B, and FIG. 8C respectively show effect of API particle size on sustained release of a. and b. (+)-TBZ polymer depot compositions (FIGS. 8A and 8B) and (+)-(α)-DHTBZ polymer depot compositions (FIG. 8C). Particle D(50) values for small (S), medium (M), and large (L) were 10-35 μm, 50-80 μm, and 100-130 μm, respectively.

FIG. 9A and FIG. 9B respectively show effect of y-irradiation (FIG. 9A) and 0.22 μm filtration (FIG. 9B) on sustained release of (+)-(α)-DHTBZ polymer depot compositions. Particle D(50) values for small (S) and large (L) were 10-35 μm and 100-130 μm, respectively.

FIG. 10: Animal PK study and PK simulations: Release of (+)-TBZ and (+)-(α)-DHTBZ from injectable (+)-TBZ polymer depot compositions following SC administration in rats and release of (+)-(α)-DHTBZ from TBZ or VBZ aqueous suspensions following oral gavage in rats. Particle D(50) values for small (S) and large (L) were 10-35 μm and 100-130 μm, respectively.

FIG. 11: Animal PK study: Release of (+)-TBZ and (+)-(α)-DHTBZ from injectable (+)-TBZ polymer depot compositions following SC administration in rats and release of (+)-(α)-DHTBZ from TBZ or VBZ aqueous suspensions following oral gavage in rats. Particle D(50) values for medium (M) was 50-80 μm.

FIG. 12: Animal PK study: Release of (+)-TBZ and (+)-(α)-DHTBZ from injectable (+)-TBZ-PLGA 88-12 polymer depot compositions following SC administration in rats and release of (+)-TBZ and (+)-(α)-DHTBZ from TBZ or VBZ aqueous suspensions following oral gavage in rats. Particle D(50) values for large (L) was 100-130 μm.

FIG. 13: Animal PK study and PK simulations: Release of (+)-(α)-DHTBZ from injectable (+)-(α)-DHTBZ polymer depot compositions following SC administration in rats and release of (+)-TBZ and (+)-(α)-DHTBZ from TBZ or VBZ aqueous suspensions following oral gavage in rats. Particle D(50) values for small (S) and large (L) were 10-35 μm and 100-130 μm, respectively.

FIG. 14: Animal PK study and PK simulations: Release of (+)-(α)-DHTBZ from injectable (+)-(α)-DHTBZ polymer depot compositions following SC administration in rats and release of (+)-TBZ and (+)-(α)-DHTBZ from TBZ or VBZ aqueous suspensions following oral gavage in rats. Particle D(50) values for small (S) and large (L) were 10-35 μm and 100-130 μm, respectively.

### DETAILED DESCRIPTION OF THE APPLICATION

It must be noted that as used herein and in the appended claims, the singular forms “a,” “an,” and “the” and similar referents are to be construed to cover both the singular and the plural, unless otherwise indicated herein or clearly contradicted by context.

As used herein, in the context of the present application, all numbers disclosed herein are approximations, whether or not the words “about” or “approximately” are used. Each numerical number means a range of the numerical value±10% of the numerical value unless otherwise indicated. For example, “about 100 mL” or “100 mL” includes any values between 90 and 110 mL.

As used herein, the term “about” or “approximately” preceding a numerical value or a series of numerical values means ±10% of the numerical value unless otherwise indicated. For example, “approximately 100 mg” means 90 to 110 mg.

Unless otherwise indicated, the term “at least” preceding a series of elements is to be understood to refer to every element in the series. Those skilled in the art will recognize or be able to ascertain using no more than routine experimentation, many equivalents to the specific embodiments of the application described herein. Such equivalents are intended to be encompassed by the application.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the application can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The term “subject” as used herein refers to any individual or patient to which the subject methods are performed. Generally, the subject is human, although as will be appreciated by those in the art, the subject may be an animal. The terms “subject” and “patient” are used interchangeably. In some embodiments, the subject is a mammal. In some embodiments, the subject is a human. In some embodiments, the subject is an animal, such as a mouse, rat, rabbit, dog, monkey, or a laboratory test animal, etc.

The present application relates to a polymeric, biodegradable, biocompatible long-acting injectable drug delivery system suitable for in-situ formation of a depot or an implant to deliver pharmaceutically active ingredients in a controlled and sustained manner. The preferred polymer depot composition of the present application is a combination of a) a VMAT2 inhibitor, including but not limited to, (3R,11bR)-tetrabenazine [(+)-TBZ, (3R,11bR)-1,3,4,6,7,11b-hexahydro-9,10-dimethoxy-3-(2-methylpropyl)-2H-benzo[a]quinolizin-2-one], (2R,3R,11bR)-dihydrotetrabenazine [(+)-(α)-DHTBZ, (2R,3R,11bR)-3-isobutyl-9,10-dimethoxy-1,3,4,6,7,11b-hexahydro-2H-pyrido[2,1-a]isoquinolin-2-ol)], (2S,3R,11bR)-dihydrotetrabenazine [(+)-(β)-DHTBZ, (2S,3R,11bR)-3-isobutyl-9,10-dimethoxy-1,3,4,6,7,11b-hexahydro-2H-pyrido[2,1-a]isoquinolin-2-ol)], a deuterated derivative thereof, a pharmaceutically acceptable salt thereof, an active metabolite thereof, or a prodrug thereof; b) one or more biodegradable, biocompatible, polymers; c) one or more pharmaceutically acceptable and biocompatible solvents; and d) one or more optional pharmaceutically acceptable excipients allowing the achievement of optimizing drug delivery.

As used herein, the term of TBZ is defined as tetrabenazine, (±)-TBZ or 1,3,4,6,7,11b-hexahydro-9,10-dimethoxy-3-(2-methoxylrpopryl)-2H-benzo(a)quinoline-2-one). It is a reversible inhibitor of vesicular monoamine transporter 2 (VMAT-2).

As used herein, the term of (+)-TBZ is defined as (+)-tetrabenazine, (3R,11bR)-TBZ, or (3R,11bR)-tetrabenazine.

As used herein, the term of (−)-TBZ is defined as (−)-tetrabenazine, (3R,11bS)-TBZ, or (3R,11bS)-tetrabenazine.

As used herein, the term of VBZ is defined as valbenazine or L-Valine, (2R,3R,11bR)-1,3,4,6,7,11b-hexahydro-9,10-dimethoxy-3-(2-methylpropyl)-2H-benzo[a]quinolizin-2-ylester.

As used herein, the term of (±)-d6-TBZ is defined as deutetrabenazine, or racemic deutetrabenazine. Deutetrabenazine is a hexahydro-dimethoxybenzoquinolizine derivative and has the following chemical name: (RR, SS)-1,3,4,6,7,11b-hexahydro-9,10-di(methoxy-d3)-3-(2-methylpropyl)¬2H-benzo[a]quinolizin-2-one. Deutetrabenazine is a racemic mixture containing RR-deutetrabenazine ((+)-d6-TBZ) and SS-deutetrabenazine ((−)-d6-TBZ).

As used herein, the term of (+)-d6-TBZ) is defined as RR-deutetrabenazine and the term of (−)-d6-TBZ is defined as SS-deutetrabenazine.

As used herein, the term of (+)-(α)-DHTBZ is defined as [+]-α-dihydrotetrabenazine, one of the metabolites of tetrabenazine.

As used herein, the term of (+)-(β)-DHTBZ is defined as [+]-β-dihydrotetrabenazine, one of the metabolites of tetrabenazine.

As used herein, the term of (−)-(α)-DHTBZ is defined as [−]-α-dihydrotetrabenazine, one of the metabolites of tetrabenazine.

As used herein, the term of (−)-(β)-DHTBZ is defined as [−]-β-dihydrotetrabenazine, one of the metabolites of tetrabenazine.

As used herein, the term of (+)-d6-(α)-DHTBZ is defined as (+)-d6-alpha-dihydrotetrabenazine, one of the metabolites of deutetrabenazine.

As used herein, the term of (−)-d6-(α)-DHTBZ is defined as (−)-d6-alpha-dihydrotetrabenazine, one of the metabolites of deutetrabenazine.

As used herein, the term of (+)-d6-(β)-DHTBZ is defined as (+)-d6-beta-dihydrotetrabenazine, one of the metabolites of deutetrabenazine.

As used herein, the term of (−)-d6-(β)-DHTBZ is defined as (−)-d6-beta-dihydrotetrabenazine, one of the metabolites of deutetrabenazine.

The present polymer depot compositions can be a viscous fluid, a solution, a gel, an emulsion, a suspension, or a semisolid dispersion that is preserved in a pre-filled syringe and ready for subcutaneous or intramuscular injection.

The polymer depot compositions can also be stabilized and filled in two separated syringes. In one syringe (A), dry powders of (+)-TBZ, (+)-(α)-DHTBZ, (+)-(β)-DHTBZ, a deuterated derivative thereof, a pharmaceutically acceptable salt thereof, an active metabolite thereof, or a prodrug thereof, is pre-filled, while the other syringe (B) is filled with a delivery vehicle that comprises one or more biodegradable, biocompatible polymers, a biocompatible organic solvent and pharmaceutical excipient(s). Prior to injection, syringes A and B are connected via a connector, followed by mixing the components thoroughly in turns of pushing the two syringe plungers back-and-forth for a sufficient number of times. Preferably, syringes A and B are male-female Luer-lock syringes that can be easily connected directly to each other and disconnected. More preferably, syringes A and B are polymer syringes that are suitable for terminal sterilization, including but not limited to E-beam, X-ray and gamma-irradiation. The final mixture for injection can be a viscous liquid, a solution, a gel, an emulsion, a suspension, or a semisolid dispersion, which is stable and ready for injection preferably within about 30 minutes and more preferably within about 1-2 hours.

The polymer depot compositions can be administrated via said syringes or devices thereof to a living subject subcutaneously, intramuscularly, intraperitoneally, or intradermally and form a depot or an implant in-situ at the injection site. As soon as the polymer depot composition comes in contact with an aqueous medium or body fluid, the biocompatible organic solvent(s) dissipates from the polymer depot composition, leaving the biodegradable, biocompatible, polymeric carrier to form a depot, or to precipitate and form a solid matrix which encapsulates the pharmaceutically active ingredients including but not limited to TBZ, (+)-TBZ, (+)-(α)-DHTBZ, (+)-(β)-DHTBZ, a deuterated derivative thereof, a pharmaceutically acceptable salt thereof, an active metabolite thereof, or a prodrug thereof.

As used herein, the term “VMAT2” is the abbreviation of vesicular monoamine transport type 2. VMAT2 inhibitors are agents that cause a depletion of neuroactive peptides, such as dopamine in nerve terminals and are used to treat chorea due to neurodegenerative diseases (such as Huntington's disease) or dyskinesia due to neuroleptic medications (tardive dyskinesia, TD). As of 2022, three VMAT2 inhibitor drug products have become available in the United States for the management of dyskinesia syndromes, each with a somewhat different spectrum of approved indications: tetrabenazine (XENAZINE® and generics: 2008), deutetrabenazine (AUSTEDO®: 2017) and valbenazine (INGREZZA®): 2017). VMAT2 inhibitors have not been associated with serum enzyme elevations during therapy or linked to instances of clinically apparent liver injury, but they have had limited general clinical use.

As used herein, a VMAT2 inhibitor includes, but is not limited to, tetrabenazine (TBZ), dihydrotetrabenazine (DHTBZ), deutetrabenazine (d6-TBZ), and deuterated dihydrotetrabenazine (d6-DHTBZ), (3R,11bR)-tetrabenazine [(+)-TBZ, (3R,11bR)-1,3,4,6,7,11b-hexahydro-9,10-dimethoxy-3-(2-methylpropyl)-2H-benzo[a]quinolizin-2-one], (2R,3R,11bR)-dihydrotetrabenazine [(+)-(α)-DHTBZ, (2R,3R,11bR)-3-isobutyl-9,10-dimethoxy-1,3,4,6,7,11b-hexahydro-2H-pyrido[2,1-a]isoquinolin-2-ol)], (2S,3R,11bR)-dihydrotetrabenazine [(+)-(β)-DHTBZ, (2S,3R,11bR)-3-isobutyl-9,10-dimethoxy-1,3,4,6,7,11b-hexahydro-2H-pyrido[2,1-a]isoquinolin-2-ol)], a deuterated derivative thereof, a pharmaceutically acceptable salt thereof, an active metabolite thereof, or a prodrug thereof.

Tetrabenazine, a hexahydro-dimethoxy-benzoquinolizine derivative, acts primarily as a reversible high-affinity inhibitor of mono-amine uptake into granular vesicles of presynaptic neurons by binding selectively to VMAT2. [Kenney C, Jankovic J. Tetrabenazine in the treatment of hyperkinetic movement disorders. Exp Rev Neurother. 2006; 6(1):7-17]. Both tetrabenazine (TBZ) and its active metabolite dihydrotetrabenazines (DHTBZ) are potent inhibitors of VMAT2.

Tetrabenazine is rapidly and extensively metabolized by first-pass metabolic reduction of the 2-keto group, generating four isomers of dihydrotetrabenazines (DHTBZ), which include (2R,3R,11bR)-DHTBZ, (2S,3S,11bS)-DHTBZ, (2S,3R,11bR)-DHTBZ, and (2R,3S,11bS)-DHTBZ. The four TBZ metabolites are likely the major pharmacologically active substances in vivo. The primary pharmacological action of TBZ and its active metabolites is to deplete the levels of monoamines (e.g. dopamine, serotonin, and norepinephrine) within the central nervous system by inhibiting the human VMAT2 [D. Scherman, B. Gasnier, P. Jaudon, J. P. Henry, Mol. Pharmacol. 33 (1988) 72-77; A. Pletscher, A. Brossi, K. F. Gey, Int. Rev. Neurobiol. 4 (1962) 275-306; A. P. Vartak, J. R. Nickell, J. Chagkutip, L. P. Dwoskin, P. A. Crooks, J. Med. Chem. 52 (2009) 7878-7882]. This transporter is predominantly expressed in the brain, which translocates monoamines from cytoplasm into synaptic vesicles, where they are both stored and protected from metabolism prior to their synaptic release. Multiple lines of evidence indicate that the binding of TBZ metabolites to VMAT2 is stereospecific [M. Kilboum, L. Lee, T. V. Borght, D. M. Jewett, K. Frey, Eur. J. Pharmacol. 278 (1995) 249e252; M. R. Kilboum, L. C. Lee, M. J. Heeg, D. M. Jewett, Chirality 9 (1997) 59e62; M. R. Kilboum, L. C. Lee, D. M. Jewett, R. A. Koeppe, K. A. Frey, J. Cereb. Blood Flow Metab. 15 (1995) S650]. Tetrabenazine enantiomers and all eight stereoisomers of dihydrotetrabenazine were synthesized and evaluated as VMAT2 inhibitors [Zhangyu Yao, Xueying Wei, Xiaoming Wu, Jonathan L. Katz, Theresa Kopajtic, Nigel H. Greig, and Hongbin Sun, European Journal of Medicinal Chemistry 46 (2011) 1841-1848]. Among the TBZ enantiomers and eight DHTBZ isomers, (+)-TBZ, (+)-(α)-DHTBZ and (+)-(β)-DHTBZ demonstrated relatively high rat VMAT2 binding affinity of 4.47, 3.96, and 13.4 nM, respectively.

As used herein, the VMAT2 inhibitor is (3R,11bR)-tetrabenazine, or (3R,11bR)-1,3,4,6,7,11b-hexahydro-9,10-dimethoxy-3-(2-methylpropyl)-2H-benzo[a]quinolizin-2-one, or (+)-TBZ.

As used herein, the VMAT2 inhibitor is referred to (2R,3R,11bR)-9,10-dimethoxy-3-(2-methylpropyl)-2,3,4,6,7,11b-hexahydro-1H-benzo[a]quinolizin-2-ol, or (2R,3R,11bR)-dihydrotetrabenazine, or (+)-α-3-isobutyl-9,10-dimethoxy-1,3,4,6,7,11b-hexahydro-2H-pyrido[2,1-a]isoquinolin-2-ol, or (+)-alpha-dihydrotetrabenazine, or (+)-(α)-HTBZ, or (+)-(α)-DTBZ, or (+)-(α)-DHTBZ. These abbreviations are used interchangeably herein. “(+)-α-DHTBZ” is one of the active metabolites of tetrabenazine.

As used herein, the VMAT2 inhibitor is (2S,3R,11bR)-1,3,4,6,7,11b-Hexahydro-9,10-dimethoxy-3-(2-methylpropyl)-2H-benzo[a]quinolizin-2-ol, or (2S,3R,11bR)-dihydrotetrabenazine, or (+)-(β)-3-isobutyl-9,10-dimethoxy-1,3,4,6,7,11b-hexahydro-2H-pyrido[2,1-a]isoquinolin-2-ol, or (+)-beta-dihydrotetrabenazine, or (+)-(β)-HTBZ, or (+)-(β)-DTBZ, or (+)-(β)-DHTBZ. These abbreviations are used interchangeably herein. “(+)-(β)-DHTBZ” is one of the active metabolites of tetrabenazine.

As used herein, deutetrabenazine is an isotopic isomer of tetrabenazine in which six hydrogen atoms have been replaced by deuterium atoms. The incorporation of deuterium slows the rate of drug metabolism and prolongs drug half-life, allowing less frequent dosing [Coppen E M, Roos R A, “Current Pharmacological Approaches to Reduce Chorea in Huntington's Disease”. Drugs. 77 (2017): 29-46]. Deutetrabenazine is extensively metabolized by the liver into active metabolites including deuterated alpha-dihydrotetrabenazine (alpha-DHTBZ) and deuterated beta-dihydrotetrabenazine (beta-DHTBZ).

The preferred VMAT2 inhibitor has low off-target binding affinity. More preferably, the VMAT2 inhibitor is (+)-TBZ, (+)-(α)-DHTBZ, (+)-(β)-DHTBZ, a deuterated derivative thereof, a pharmaceutically acceptable salt thereof, an active metabolite thereof, or a prodrug thereof. The deuterated derivatives include deuterated TBZ, deuterated (+)-TBZ, deuterated (+)-(α)-DHTBZ, deuterated (+)-(β)-DHTBZ, and the like.

In a preferred embodiment, the VMAT2 inhibitor is (+)-TBZ. (+)-TBZ is optically purified from racemic TBZ where the other stereoisomer (−)-TBZ is removed. Racemic TBZ can be rapidly metabolized to its four reduced form (+)-(α)-DHTBZ, (−)-(α)-DHTBZ, (+)-(β)-DHTBZ and (−)-(β)-DHTBZ in vivo. Among those, (−)-(α)-DHTBZ and (−)-(β)-DHTBZ are likely to be responsible for the cause of serious side effects due to high alterative binding to dopamine D2s and serotonin 5-HT receptors. In this particular embodiment, using optically pure (+)-TBZ as the only pharmaceutically active ingredient would significantly lower the risk of severe side effects generated from off-target binding, which provides a much preferred and safer drug product.

In another preferred embodiment, VMAT2 inhibitor is (+)-(α)-DHTBZ or (+)-(β)-DHTBZ. Both (+)-(α)-DHTBZ and (+)-(β)-DHTBZ are the reduced forms of (+)-TBZ. (+)-(α)-DHTBZ and (+)-(β)-DHTBZ can be generated from (+)-TBZ in vivo majorly in the liver by carbonyl reductase or, can also be easily synthesized by a person of ordinary skill in the art. Instead of the parent compound, a single active metabolite can further guarantee minimal metabolism variation between patients (especially for patients with CYP 2D6 polymorphism) that can generate additional complications while receiving VMAT2 inhibitors.

The polymer depot composition of the present application is produced by combining a VMAT2 inhibitor including (+)-TBZ, (+)-(α)-DHTBZ, (+)-(β)-DHTBZ, a deuterated derivative thereof, a pharmaceutically acceptable salt thereof, an active metabolite thereof, or a prodrug thereof with a solution of a solid, biodegradable, biocompatible polymer dissolved in one or more pharmaceutically acceptable and biocompatible solvents. The polymer depot composition can be administered by a syringe and a needle to a patient in need of treatment. Any suitable biodegradable polymer can be employed, provided that the biodegradable polymer is at least substantially insoluble in body fluid.

The application is based in part on the discovery that incorporation of a VMAT2 inhibitor in a viscous depot vehicle produces a formulation that has low initial burst release, minimal lag time, and near zero-order release in vivo. For a depot formulation, this release profile is surprising because the evidence in the art is that a low burst, near zero-order release is virtually impossible to attain unless special steps are taken, such as coatings for drugs and microencapsulation.

The polymer depot composition according to embodiments of the application can be prepared as injectables. The administration route may include a subcutaneous, intramuscular, intramyocardial, adventitial, intratumoral, or intracerebral. Multiple or repeated injections may be administered to a subject to maintain therapeutic effect or to the subject that requires further administration of the drug for any reason. The polymer depot composition serves as an implanted sustained release drug delivery system after injection into the subject. Such controlled release can be over a period of one week, more than one week, one month, or more than one month. Preferably, the controlled release is over at least a period of one week, more preferably over a period of at least one month.

In certain embodiments of the application, the viscous depot vehicle includes a biocompatible polymer, i.e., a polymer that would not cause irritation or necrosis in the tissues of the subjects. The biocompatible polymers of the application may be bioerodible, i.e., gradually decompose, dissolve, hydrolyze and/or erode in situ. Examples of bioerodible polymers include, but are not limited to, polylactides, polyglycolides, polycaprolactones, polyanhydrides, polyamines, polyurethanes, polyesteram ides, polyorthoesters, polydioxanones, polyacetals, polyketals, polycarbonates, polyorthocarbonates, polyphosphazenes, poly(malic acid), poly(amino acids), polyvinylpyrrolidone, polyethylene glycol, polyhydroxycellulose, polysaccharides, chitin, chitosan, and copolymers, terpolymers and mixtures thereof. The polymer is dissolved in a pharmaceutically acceptable solvent and is typically present in the solution in an amount ranging from about 5 to 80% by weight, preferably from about 20 to 70%, often more preferably from about 30 to 65% by weight.

In one embodiment, the biocompatible polymer is a polylactide. A polylactide polymer is a polymer based on lactic acid. The term “lactic acid” as used herein includes the isomers L-lactic acid, D-lactic acid, DL-lactic acid, L-lactide, D-lactide, and DL-lactide. Polylactide, also known as poly(lactic acid) or polylactic acid (abbreviation PLA), is a thermoplastic polyester with backbone formula (C₃H₄O₂)n or [—C(CH₃)HC(═O)O—]n, formally obtained by condensation of lactic acid C(CH₃)(OH)HCOOH by removing water (H₂O). It can also be prepared by ring-opening polymerization of lactide [—C(CH₃)HC(═O)O—]₂, the cyclic dimer of the basic repeating unit. Polylactide contains an asymmetric a-carbon which is typically described as the D or L form in classical stereochemical terms and sometimes as R and S form, respectively. The enantiomeric forms of the polymer PLA are poly D-lactic acid (PDLA) and poly L-lactic acid (PLLA). The term “polylactide” as used herein includes poly(L-lactic acid), poly(D-lactic acid), poly(DL-lactic acid), poly(L-lactide), poly(D-lactide), and poly(DL-lactide).

In another embodiment of the application, the biocompatible polymer is a poly(lactide-co-glycolide), a copolymer based on lactic acid and glycolic acid. PLGA or PLG is generally an acronym for poly(D,L-lactide-co-glycolide) or poly(D,L-lactic-co-glycolic acid) where D- and L-lactic acid forms are in equal ratio. The term “glycolic acid” as used herein includes glycolide. PLGA is synthesized by ring-opening co-polymerization of two different monomers, the cyclic dimers (1,4-dioxane-2,5-diones) of glycolic acid and lactic acid. Polymers can be synthesized as either random or block copolymers thereby imparting additional polymer properties. Common catalysts used in the preparation of this polymer include tin(II) 2-ethylhexanoate, tin(II) alkoxides, or aluminum isopropoxide. During polymerization, successive monomeric units (of glycolic or lactic acid) are linked together in PLGA by ester linkages, thus yielding a linear, aliphatic polyester as a product [Astete, C. E. & Sabliov, C. M. (2006). “Synthesis and characterization of PLGA nanoparticles”. Journal of Biomaterials Science, Polymer Edition. 17 (3): 247-289].

PLGA is a linear copolymer that can be prepared at different ratios between its constituent monomers, lactic (LA) and glycolic acid (GA). Depending on the ratio of lactide to glycolide used for the polymerization, different forms of PLGA can be obtained: these are usually identified in regard to the monomers' ratio used (i.e., PLGA 75:25 identifies a copolymer consisted of 75% lactic acid and 25% glycolic acid). The crystallinity of PLGAs vary from fully amorphous to fully crystalline depending on block structure and molar ratio. PLGAs typically show a glass transition temperature in the range of 40-60° C. PLGA can be dissolved by a wide range of solvents, depending on composition.

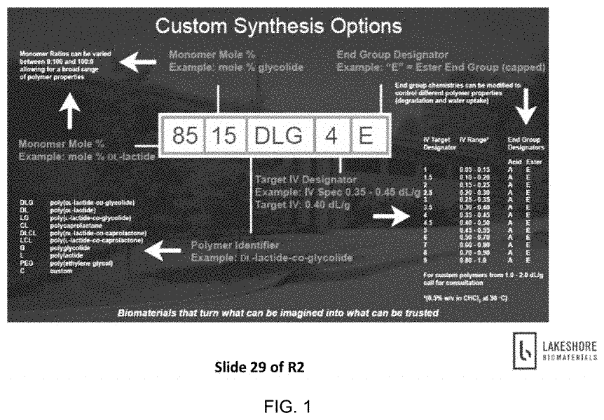

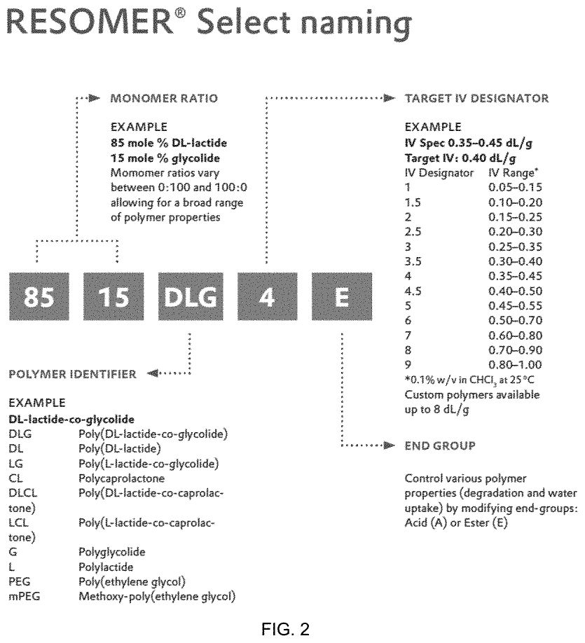

The poly(D,L-lactide-co-glycolide) and ploy(D,L-Lactide) used herein can be purchased from various suppliers such as Evonik and Ashland. The naming of various polymers was first published in presentation slide 29 by John Middleton of Lakeshore Biomaterials in 2007 (see reference “Tailoring of Poly(lactide-co-glycolide) to Control Properties” at: https://mafiadoc.com/tailoring-of-polylactide-co-glycolide-to-control-_59c989c41723dde2802d6956.html). In 2018, Evonik published “RESOMER® product brochure” including the “Resomer° Select naming” as shown in FIGS. 1 and 2.

PLGA or PLA degrades by hydrolysis of its ester linkages in the presence of water. It has been shown that the time required for degradation of PLGA is related to the monomers' ratio in the PLGA: the higher the content of glycolide units, the shorter the time required for degradation as compared to predominantly lactide materials, PLA. In addition, polymers that are end-capped with esters (as opposed to the free carboxylic acid) demonstrate longer degradation half-lives [Samadi, N.; Abbadessa, A.; Di Stefano, A.; van Nostrum, C. F.; Vermonden, T.; Rahimian, S.; Teunissen, E. A.; van Steenbergen, M. J.; Amidi, M. & Hennink, W. E. (2013). “The effect of lauryl capping group on protein release and degradation of poly(D,L-lactic-co-glycolic acid) particles”. Journal of Controlled Release. 172 (2): 436-443]. This flexibility in degradation has made it convenient for fabrication of many medical devices, such as, grafts, sutures, implants, prosthetic devices, surgical sealant films, micro and nanoparticles [Pavot, V; Berthet, M; Rességuier, J; Legaz, S; Handké, N; Gilbert, S C; Paul, S; Verrier, B (December 2014). “Poly(lactic acid) and poly(lactic-co-glycolic acid) particles as versatile carrier platforms for vaccine delivery”. Nanomedicine (Lond.). 9 (17): 2703-18].

In certain embodiments of the application, the PLGA polymers may have a lactic-acid to glycolic-acid monomer ratio of from about 100:0 to 50:50, preferably about 85:15 (75:25 to 95:5), about 75:25 (65:35 to 85:15), about 65:35 (55:45 to 75:25), and about 50:50 (40:60 to 60:40). The PLGA polymer has a weight average molecular weight (Mw) ranging from about 1,000 to about 120,000, preferably from about 5,000 to about 40,000, as determined by gel permeation chromatography (GPC). Further preferably, the PLGA polymer is synthesized with a monoalcohol such as ethanol or dodecanol to obtain a PLGA polymer having one ester terminal functional group and one hydroxyl end group. The PLGA polymer can also be synthesized with a diol such as propylene-1,3-diol, 1,4-butanediol, 1,5-pentanediol, 1,6-hexanediol, 1,10-decanediol to obtain a PLGA polymer having one hydroxyl group at each end of the polymer. The PLGA polymer can also be made to have one or two carboxyl terminal groups. Preferably, the PLGA polymer is practically insoluble in aqueous medium or in body fluid, yet is readily soluble or miscible in biocompatible organic solvents to form a solution, or a viscous fluid.

In still another embodiment, the desired biodegradable, biocompatible, polymeric carrier is, but not limited, to poly lactic-co-glycolic acid (PLGA) and poly lactic acid (PLA). Both PLGA and PLA are insoluble in water, but have certain solubility in biocompatible solvents or a combination of solvents. Once dissolved in such biocompatible solvents or a combination thereof, viscous delivery vehicles can be formed. The delivery vehicles can subsequently be formulated with pharmaceutically active ingredients to form the polymer depot compositions of the application. As soon as the polymer depot composition comes in contact with an aqueous medium or body fluid, the biocompatible organic solvents dissipate from the polymer depot composition, leaving the biodegradable, biocompatible polymer to form a gel depot, or to precipitate and form a solid matrix which encapsulates the VMAT2 inhibitors such as (+)-TBZ, (+)-(α)-DHTBZ, (+)-(β)-DHTBZ, a deuterated derivative thereof, a pharmaceutically acceptable salt thereof, an active metabolite thereof, or a prodrug thereof, which is then released in a controlled and sustained manner for a duration of at least one week and more preferably of a least one month.

In one embodiment, the PLGA polymers are supplied by Evonik Industries. Some of the examples of Resomer polymers are shown in the Table below.

| Product Number | Product Description |
|---|---|
| Resomer ® R 202 H | Poly(D,L-lactide) acid terminated, M_{w} 10,000-18,000 |
| Resomer ® R 203 H | Poly(D,L-lactide) acid terminated, M_{w} 18,000-24,000 |
| Resomer ® L 206 S | Poly(L-lactide), ester terminated |
| Resomer ® R 202 S | Poly(D,L-lactide), ester terminated, M_{w} 10,000-18,000 |
| Resomer ® R 203 S | Poly(D,L-lactide), ester terminated, M_{w} 18,000-28,000 |
| Resomer ® RG 502 | Poly(D,L-Lactide-co-Glycolide), lactide:glycolide 50:50, ester |
| | terminated, M_{w} 7,000-17,000 |
| Resomer ® RG 502 H | Poly(D,L-lactide-co-glycolide), lactide:glycolide 50:50, acid |
| | terminated, M_{w} 7,000-17,000 |
| Resomer ® RG 503 | Poly(D,L-lactide-co-glycolide), lactide:glycolide 50:50, ester |
| | terminated, M_{w} 24,000-38,000 |
| Resomer ® RG 503 H | Poly(D,L-lactide-co-glycolide), acid terminated, lactide:glycolide |
| | 50:50, M_{w} 24,000-38,000 |
| Resomer ® RG 504 | Poly(D,L-lactide-co-glycolide), lactide:glycolide 50:50, ester |
| | terminated, M_{w} 38,000-54,000 |
| Resomer ® RG 504 H | Poly(D,L-lactide-co-glycolide), acid terminated, lactide:glycolide |
| | 50:50, M_{w} 38,000-54,000 |
| Resomer ® RG 505 | Poly(D,L-lactide-co-glycolide), lactide:glycolide 50:50, ester |
| | terminated, M_{w} 54,000-69,000 |
| Resomer ® RG 653 H | Poly(D,L-lactide-co-glycolide), lactide:glycolide 65:35, acid |
| | terminated, M_{w} 24,000-38,000 |
| Resomer ® RG 752 H | Poly(D,L-lactide-co-glycolide), acid terminated, lactide:glycolide |
| | 75:25, M_{w} 4,000-15,000 |
| Resomer ® RG 756 S | Poly(D,L-lactide-co-glycolide), ester terminated, lactide:glycolide |
| | 75:25, M_{w} 76,000-115,000 |
| Resomer ® RG 858 S | Poly(D,L-lactide-co-glycolide), ester terminated, lactide:glycolide |
| | 85:15, M_{w} 190,000-240,000 |
| Resomer ® X 206 S | poly(dioxanone) |

The pharmaceutically acceptable and biocompatible solvents in the present application are water soluble, miscible to dispersible or at least showing partial solubility in water. As used herein, the terms “soluble” and “miscible” are meant to be used interchangeably. When combined with biodegradable, hydrophobic polymers, the solvents can readily solvate the said polymers, resulting in delivery vehicles with desired viscosity. The delivery vehicles can be further formulated with pharmaceutically active ingredients to form the polymer depot compositions of the application to achieve controlled and sustained drug delivery. Examples of the pharmaceutically acceptable and biocompatible solvents include, but are not limited to, ethanol (EtOH), 1-Methyl-2-pyrrolidone or N-methyl-2-pyrrolidone (NMP), benzyl benzoate (BB), benzyl alcohol (BA), dimethyl sulfoxide (DMSO), tetraglycol (or glycofurol), dimethylacetamide (DMAc), triacetin (TA), low molecular weight polyethylene glycol (i.e. PEG 300 and PEG 400), polyethylene glycol esters, methyl acetate, ethyl acetate, ethyl oleate, glycerol, esters of caprylic and/or capric acids with glycerol or alkylene glycols, and the combination thereof.

In one preferred embodiment, the pharmaceutically acceptable and biocompatible solvent is N-Methyl-2-pyrrolidone (NMP).

According to the present application, the polymer depot composition comprises one biodegradable, biocompatible polymer and one pharmaceutically acceptable solvent to form the delivery vehicle. Preferably, the biodegradable, biocompatible polymer is substantially water-insoluble, which precipitates or forms a water-insoluble depot or implant after injection. In a preferred embodiment, PLGA as defined herein is used to prolong the release of VMAT2 inhibitors such as (+)-TBZ, (+)-(α)-DHTBZ, (+)-(β)-DHTBZ, a deuterated derivative thereof, a pharmaceutically acceptable salt thereof, an active metabolite thereof, or a prodrug thereof. In one embodiment, the polymer depot composition comprising 30% (+)-TBZ suspended in a polymer solution of RG502/NMP at 45/55 w/w ratio demonstrated about 40% cumulative release of (+)-TBZ over 3 weeks in vitro, while the polymer depot composition comprising 30% (+)-TBZ suspended in a polymer solution of RG503/NMP at 35/65 w/w ratio showed just over 20% accumulated release of (+)-TBZ over 3 weeks in vitro. Furthermore, release duration could be further prolonged by using PLA to replace PLGA. In another embodiment, the polymer depot composition comprising 30% (+)-TBZ suspended in a polymer solution of PLA/NMP at 60/40 w/w ratio demonstrated less than 20% accumulated drug release over 3 weeks in vitro.

According to the present application, the controlled and sustained delivery of (+)-(α)-DHTBZ can also be achieved. In one embodiment, the polymer depot composition comprising 30% (+)-(α)-DHTBZ suspended in a polymer solution of RG502/NMP at 65/35 w/w ratio showed drug release in a sustained manner with about 70% accumulated release over 3 weeks. In another embodiment, the polymer depot composition comprising 30% (+)-(α)-DHTBZ suspended in a polymer solution of RG503/NMP at 45/55 w/w ratio displayed less than 40% accumulated release over 3 weeks in vitro. In all these embodiments, the polymer depot compositions are capable of forming a depot/implant at the injection site upon administration to a living subject. The inventive compositions are competent for maintaining plasma concentrations of (+)-TBZ, (+)-(α)-DHTBZ and (+)-(β)-DHTBZ at or above therapeutic level preferably for 1 to 2 weeks, more preferably for 2 to 4 weeks, and most preferably for 1 to 3 months with minimum variation in plasma concentration and narrow peak to trough (P/T) ratio.

According to the present application, the sustained release profile of VMAT2 inhibitor is adjustable. Factors impacting release profiles of VMAT2 inhibitors include, but not limited to, type of the biodegradable polymers, end function groups of the biodegradable polymers (ester terminated or carboxylic acid terminated or hydroxyl terminated), polymer molecular weight (Mw) and Mw distribution, type of the biocompatible solvents or the combination thereof, ratio of biodegradable polymer to biocompatible solvent, type of the VMAT2 inhibitor ((+)-TBZ or (+)-DHTBZ), drug loading, as well as particle size of the VMAT2 inhibitors. In some embodiments, various types of biodegradable polymers including but not limited to DL-lactide/glycolide copolymer at about 50:50 ratio, DL-lactide/glycolide copolymer (PLGA) at about 50:50 ratio with acid terminal, DL-lactide/glycolide copolymer at about 75:25 ratio, DL-lactide/glycolide copolymer at about 75:25 ratio with acid terminal, DL-lactide/glycolide copolymer at about 88:12 ratio, and poly (DL-lactide) (PLA) were selected to make polymer solution vehicles in NMP. The sustained release compositions were obtained by controlling formulation parameters including, but not limited to, a ratio of the biodegradable polymer to biocompatible solvent, type of beneficial pharmaceutically acceptable excipients, type of the VMAT2 inhibitors, drug loading, as well as particle size of the VMAT2 inhibitors.

The ratio of polymer to biocompatible solvent could be one of critical factors affecting release profiles of in-situ forming depot drug delivery systems. However, it was found that the correlation between initial burst release of VMAT2 inhibitors and polymer/solvent ratio is not straightforward. In one embodiment, (+)-(α)-DHTBZ showed reduced initial release from PLGA/NMP in situ forming depot as polymer content increased. When (+)-(α)-DHTBZ drug loading is fixed at 30%, changing the ratio of RG502/NMP from 65/35 to 30/70 w/w resulted in about 4% and about 18% initial drug release, respectively; while changing the ratio of RG503/NMP from 50/50 to 45/55 w/w resulted in about 5% and about 8% initial drug release, respectively. Surprisingly, in another embodiment, the polymer to biocompatible solvent ratio in the in situ forming drug delivery system showed no impact on initial release of (+)-TBZ. When drug loading is fixed at 20%, changing the ratio of RG502/NMP from 45/55 to 35/65 w/w resulted in the same level of initial drug release of about 10%. In addition, while drug loading is fixed at 30%, changing the ratio of RG503/NMP from 35/65, 25/75, and 15/85 w/w resulted in the same level of initial drug release of about 4%. These results are unexpected since in general, reduced polymer to biocompatible solvent ratio would lead to a higher initial burst due to reduced viscosity of the polymer solution.

Biocompatible solvent or a combination of biocompatible solvents can have a major influence on long-acting sustained drug delivery. Wang et al. developed a sustained release system composed of hydrophilic solvent-induced PLGA based in situ forming systems. They investigated the factors affecting drug release including the effect of biocompatible solvent(s). The initial release was reduced 3.7-8.0 times and the plasma level were significantly prolonged from 4 days to 10-15 days as the hydrophilic NMP was replaced by the hydrophobic co-solvent composed of 90% benzyl benzoate (BB) and 10% co-solvent (benzyl alcohol, triacetin, or NMP) (Wang et al., RSC Adv., 2017, 7, 5349-5361). In a completely opposite way, we found replacement of NMP by a small portion of BB actually resulted in similar initial release, followed by a faster accumulated release. In one embodiment, 40% (+)-(α)-DHTBZ was mixed with RG502/BB/NMP (65/5/30) to form a depot composition and tested for in vitro release. The results showed substantially faster release when only 5% (w/w) of the NMP in the polymer solution was substituted with BB (14-day release increased from about 50% to about 70%). In another embodiment, 50% (+)-(α)-DHTBZ was mixed with RG503H/BB/NMP at 50/5/45 w/w ratio to form a polymer depot composition and tested for in vitro release. The results also demonstrated accelerated release after replacing 5% (w/w) of NMP in the polymer solution vehicle by BB (14-day release increase from about 20% to about 30%). In addition, a similar trend was found when using (+)-TBZ as the therapeutic agent. In one embodiment, 50% (+)-TBZ was mixed with a polymer solution vehicle composed of RG503/NMP at 45/55 w/w ratio to form a polymer depot composition and tested for in vitro release. While the initial release was the same, the results showed faster overall release when only 5% (w/w) of the NMP in the polymer solution was substituted by BB (21-day release increased from about 20% to about 30%). These findings were unexpected to what the prior arts in the related field have revealed. Furthermore, such method we demonstrated here in the present application provides an advantage on the controlling overall release profile of VMAT2 inhibitors without affecting the initial release, which is quite challenging to achieve most of the time when developing a sustained-release, in situ forming depot drug delivery system.

In certain embodiments, high drug loading (DL %) is desired for less potent drugs in long-acting sustained delivery systems as it is key to maintain the injection volume within a reasonable range. However, DL % can also alter the release profile. Higher drug loading is usually accompanied with increased burst release in the ATRIGEL® or its related drug delivery systems. Geng and his team developed an in situ forming gel based on PLA matrix depot for sustained release of Ivermectin. They found the release rate of Ivermectin was positively correlated to its DL %. Cumulative release was increased 2.4-2.9 and 3.1-3.7 times as Ivermectin loading was increased from 1% to 2% and 1% to 4%, respectively (Geng et al., International Journal of Biological Macromolecules Vol. 85, April 2016, 271-276). The prior art seems to imply such a simple positive correlation between DL % and drug release, but in one embodiment we unexpectedly found that the effect of DL % on (+)-TBZ and (+)-(α)-DHTBZ release was far more complicated. For example, in formulations composed of (+)-TBZ suspended in a polymer solution vehicle composed of RG503/NMP at 35/65 w/w ratio at various DL %, while the cumulative release increased with escalating DL %, the initial release was almost identical at about 3% for drug loading at 20, 30, and 50%. However, for the same formulation composition but reduced drug loading at 5%, significantly higher initial burst release (over 10%) was found even with larger API particles. In another embodiment, the initial release was almost identical at about 3-5% for drug loadings at 50, 60, and 70% in formulations composed of (+)-TBZ suspended in a polymer solution vehicle composed of RG752H/NMP at 50/50 w/w ratio. On the other hand, for (+)-(α)-DHTBZ, the DL % affects the release profile even more differently. For example, while the initial burst release did not change with varying DL %, the overall release rate was accelerated from 15% to 25% when the DL % was increased in the polymer solution vehicle composed of RG503/NMP at 50/50 w/w ratio. Whereas, the release profile was almost identical for formulations composed of the same polymeric vehicle at both 30 and 40% drug loading. What is more exceptional, varying the DL % worked completely opposite to carboxylic acid-terminated RG503H in combination with (+)-(α)-DHTBZ. The release of (+)-(α)-DHTBZ was indeed slower with increasing DL % in the RG503H formulations. These findings once again, reemphasized that the effect of drug loading on release profiles of VMAT2 inhibitor cannot be managed by simply mimicking or reproducing formulations in other related prior arts disclosed elsewhere.

It is clear that, to develop a sustained-release, in situ forming depot delivery system for VMAT2 inhibitor, a person of ordinary skill in the art cannot simply rely on the known information disclosed in other prior arts to achieve the desired release profile.

Generally speaking, particle size can alter the release profile in suspension formulations (Drug Des. Devel. Ther. 2013; 7: 1027-1033.). Dissolution rate is positively correlated to the surface area of the particles in a suspension formulation. While specific surface area increases with decreasing particle size of the drug, so does the drug dissolution rate. A substantial difference in dissolution rate can exist according to the variation on particle size and the relative surface area, especially during the initial period of the dissolution. In the present application, we tailored API particle size as an effective approach on tuning for desirable release profiles for VMAT2 inhibitors. Surprisingly, the impact of API particle size on drug release was far more complicated and could not be simply applied from one type of VMAT2 inhibitor to another. In one embodiment, small (+)-TBZ particles (D50˜50 μm) demonstrated higher initial release and faster accumulated release, compared to large (+)-TBZ particles (D50, ˜100 μm) from the formulations composed of RG502/NMP at 60/40 w/w ratio with 50% drug loading and RG503/NMP at 35/65 w/w ratio with 30% drug loading. On the other hand, if replacing the regular, ester-terminated polymer by carboxylic acid-terminated polymers, the effect of (+)-TBZ particle size on release disappeared (30% drug loading in a polymer solution vehicle composed of RG503H/NMP at 35/65 w/w ratio). In another embodiment, smaller (+)-TBZ particles also resulted in limited effect on in vitro release from a polymer solution composed of RG752H/NMP at 55/45 w/w ratio, regardless if the DL % was 60 or 70%. Therefore, the results we discovered from carboxylic acid-terminated PLGA polymers were unique. Carboxylic acid-terminated PLGA polymers have been utilized in some approved drug products for 1-month delivery (i.e. PERSERIS®, PLGH 8020) due to its faster polymer degradation, compared to regular, ester-terminated PLGA polymers. Taking advantage of carboxylic acid-terminated PLGA polymers in eliminating API particle size variation on the release profile is novel and hasn't been disclosed in other prior arts. Furthermore, since batch-to-batch API particle size variation can be a hurdle from a product development point of view, what we disclosed in the present application can be of great merit on producing consistent drug products with a reproducible release profile.

According to the present application, we also enabled terminal sterilization processes for sustained release formulations composed of biodegradable, polymeric vehicles and VMAT2 inhibitor. Gamma-irradiation is one of the most widely adopted terminal sterilization process for injectable drug products and medical devices. However, it is well known that polymer properties, such as polymer molecular weight (Mw), can be substantially changed after exposure to gamma-ray, while the change on polymer Mw can significantly alter drug release profile. Shapourgan and co-workers investigated the effect of gamma-irradiation on the release profile of leuprolide acetate from PLGA-based in situ forming system. A decreased glass transmission temperature (Tg) of PLGA from 43.4 to 38.1° C. after gamma-irradiation at 8 kGy was observed. PLGA Mw was also reduced by more or less 18% post gamma-irradiation. Furthermore, post gamma-irradiated PLGA matrices showed higher porosity than the non-irradiated PLGA matrices. These impacts together, led to faster release of leuprolide acetate from gamma-irradiated PLGA in situ forming depot, compared to the non-irradiated PLGA matrices (Shapourgan et al., Curr Drug Deliv. 2017; 14(8): 1170-1177). In one embodiment, gamma sterilization (25-40 kGy) was investigated for some viscous (+)-(α)-DHTBZ polymer suspensions. Unexpectedly, while accelerated release was found from post-irradiated formulations composing of (+)-(α)-DHTBZ suspended in a polymer solution vehicle composed of RG503/NMP at 50/50 w/w ratio with 40% drug loading and (+)-(α)-DHTBZ suspended in a polymer solution vehicle composed of RG503H/NMP at 50/50 w/w ratio with 50% drug loading, the release profiles for (+)-(α)-DHTBZ suspended in a polymer solution vehicle composed of RG502H/NMP at 60/40 w/w ratio with 40% drug loading almost did not change after the gamma irradiation process.

Furthermore, alternative approaches, such as filtration through 0.22 μm filter could be another option for terminal sterilization for low viscosity, polymer-based, in situ forming depot drug delivery systems. However, in order to provide long-term release in a sustained manner, PLGA or PLA-based formulations generally are viscous solutions or suspensions, which makes filtration very challenging. In one embodiment, a (+)-(α)-DHTBZ polymer depot formulation made of RG502/NMP at 40/60 w/w ratio was prepared with 23% drug loading. Filtration of such vehicle through a 0.22 μm disc filter was easy and straightforward. In vitro release profile of the formulations made of filtered and non-filtered polymer solution vehicle was identical, which demonstrated the feasibility of using 0.22 μm filtration as the terminal sterilization process for those formulations composed of less viscous polymer solution vehicles. In the present application, we demonstrated that either 0.22 μm filtration or gamma irradiation at 25-40 kGy could be an optional terminal sterilization process for the proposed VMAT2 inhibitor polymer suspensions.

The present application further provides methods of preparing and using such polymer depot compositions. In one embodiment, a method of preparing such compositions comprising of (+)-TBZ, (+)-(α)-DHTBZ, (+)-(β)-DHTBZ, a deuterated derivative thereof, a pharmaceutically acceptable salt thereof, an active metabolite thereof, or a prodrug thereof, one or more biocompatible organic solvents, and one or more pharmaceutically acceptable polymeric, water-insoluble carriers. Preferably, the pharmaceutically acceptable polymeric, water-insoluble carrier is dissolved, or mixed with the biocompatible organic solvents to form the delivery vehicle first, followed by dissolving or suspending (+)-TBZ, (+)-(α)-DHTBZ, (+)-(β)-DHTBZ, a deuterated derivative thereof, a pharmaceutically acceptable salt thereof, an active metabolite thereof, or a prodrug thereof in the delivery vehicle. The present inventive polymer depot composition can be a viscous fluid, semi-solid, or uniform suspensions ready for injection in a pre-filled syringe. The preferred composition can also be a homogeneous, viscous fluid, semi-solid, or uniform suspensions after adequate mixing prior to injection. Such compositions are physio-chemically stable prior to and during the preparation process. Preferably, such compositions are stable during manufacturing, sterilization, storage, and subsequent administration to a living subject. The polymer depot composition is preferably to be administrated via syringes or similar devices thereof to a living subject subcutaneously, intramuscularly, intraperitoneally, or intradermally and form an in-situ forming depot or implant. Preferably, the polymer depot composition of the present application has an initial release in vivo no more than 30% within 24 hours, more preferably no more than 20% within 24 hours, most preferably, no more than 10% within 24 hours. With the desired components, the polymer depot composition can sustainably deliver the pharmaceutical active ingredient above the therapeutic level preferably for 1 to 2 weeks, more preferably for 2 to 4 weeks, and most preferably for 1 to 3 months with minimum variation in plasma concentration and narrow P/T ratio (preferably from 1-10 and more preferably from 1-4, and still more preferably from 1 to 2), which can surely help limit potential side effects so as to provide an improved safety profile for patients. The polymer depot compositions are biocompatible and degrade in a living subject and can be absorbed by the body after drug delivery is done.

### EXAMPLES

The following examples demonstrate the compositions and methods of the present application. The following examples should not be considered as limitations, but should merely teach the skill in the art how to make the effective sustained release injectable polymer depot compositions.

### HPLC Analytical Method

A calibration curve was obtained through the HPLC method below to quantify the concentration of (+)-TBZ and/or (+)-DHTBZ in a sample with unknown API content.

### Materials

### Reagents

Mill-Q water, resistivity greater than 18.0 MΩ-cm, or equivalent.

Ammonium acetate, ACS grade or equivalent

Sodium hydroxide, ACS grade or equivalent

Methanol (MeOH), HPLC grade

Isopropyl alcohol (IPA), HPLC grade

N-Methyl-2-pyrrolidone (NMP), HPLC grade

### Reference Standards

(+)-TBZ API with the defined potency

### Instruments and Parameters

### HPLC

Shimadzu HPLC System:

Binary Pump: Model LC-20AT

Degasser: Model DGU-20A3R

Autosampler: Model SIL-30A HT

Column Oven: Enshine, Super CO-150 (non-Shimadzu)

Detector: Model SPD-20A

### Parameters

Column: XBridge C18 Column, 5 μm 4.6×150 mm

Mobile Phase A: 10 mM ammonium acetate, pH 6.8±0.1;

B: MeOH

Isocratic mode: NB=30/70

Flow rate: 1 mL/min

Column temp: 40° C.

Injection vol: 2 μL

Detection: 214 nm

Run time: 8 min

### Sample Preparation

### Mobile Phase A

Dissolve about 0.77 g of ammonium acetate in 1000 mL water, adjust pH to 6.8 ±0.1 with 0.1N sodium hydroxide aqueous solution. Filter through 0.22 μm PTFE membrane filter and degas before use.

### Sample Solvent

Isopropyl alcohol

### Standard Solution

Accurately weigh 20±1 mg of (+)-TBZ Reference Standard into a 20 mL volumetric flask, add 10 mL of sample solvent to dissolve it, dilute to the volume with sample solvent and mix well. Dilute this solution with sample solvent to obtain standard solutions at 2, 5, 10, 50, 100, 200, and 500 μg/mL.

### Sample Solution

### For (+)-TBZ API Samples (0.1 mg/mL of TBZ)

Accurately weigh 10 mg of API sample into a 10 mL volumetric flask, add 5 mL of sample solvent to dissolve it, dilute to the volume with sample solvent and mix well. Pipette 1 mL of above solution into a 10 mL volumetric flask, dilute with sample solvent to volume, and mix well.

### For (+)-TBZ Drug Product Samples (0.1 mg/mL of TBZ)

Accurately weigh 40 mg of drug product (PLGA or PLA-containing formulation, assuming drug loading is 50%, w/w) sample into a 20 mL volumetric flask, add 15 mL of NMP to dissolve it, dilute to the volume with NMP and mix well. Take 1 mL of the sample solution prepared above, added into a 10 mL volumetric flask, add 5 mL of IPA to dilute it, dilute to the volume with IPA and mix well. Vortex the solution followed by centrifugation at 12000 rpm for 3 minutes to aggregate the precipitate. The supernatant is then filtered through a 0.22 μm PTFE filter (discard the initial 2 mL) and transferred to an HPLC vial for injection.

### GPC Analytical Method

Polymer MW was analyzed via gel permeation chromatography (GPC, also called size exclusion chromatography, SEC) as one key parameter for polymer selection on formulation development in this application.

### Materials

### Reagents

Tetrahydrofuran (THF), stabilized, HPLC grade.

N-methyl-2-pyrrolidone (NMP), pharma grade or ACS reagent.

### GPC Standards

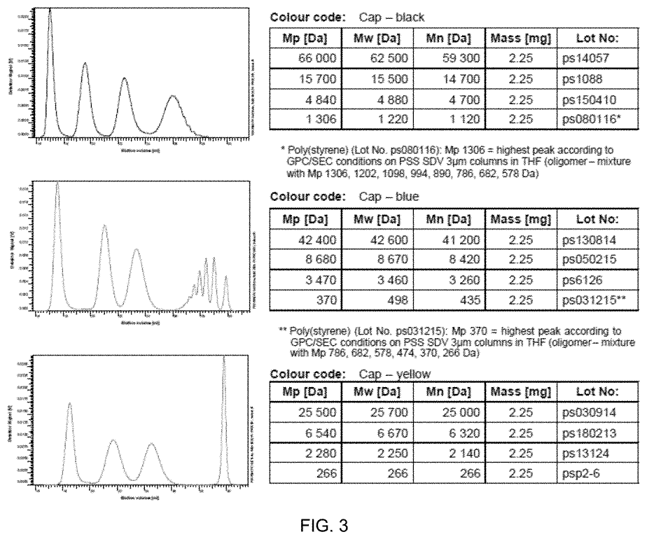

GPC calibration kits: Pskitr1L ReadyCal-Kit (polystyrene), Mp: 266-66,000 Da are purchased from PSS-Polymer Standards Service USA Inc. Molecular weight information from the official document of ReadyCal-Kit, PSS-pskitr1l are listed in FIG. 3. Each vial of standards contains four polystyrene standards with different Mp.

### Instruments and Parameters

### GPC System

Shimadzu Nexera HPLC system composed of: Degasser, model DGU-20A 5R, Binary pump, model LC-30AD, RI detector, model RID-10A, Autosampler, model SIL-30AC, Column oven, model CTO-20AC

Software: LabSolutions

### GPC Column

Two Agilent ResiPore (#1113-6300) 300×7.5 mm, 3 μm particle size columns in series.

### GPC Condition

Mobile Phase/Sample buffer: THF (stabilized).

Flow rate: 1 mL/min.

Column temp: 40° C.

Injection volume: 50 μL.

Run time: 30 minutes.

Reflective Index Detector:

Polarity: positive

Temperature: 40° C.

Response: 1.5 sec

Sample concentration: 2 mg (polymer)/mL in THF.

### Sample Preparation

### Standard Preparation

Prepare molecular weight standards (polystyrene) following the official instructions for PSS-pskitr1l ReadyCal-Kit. Add 1 mL of THF into each vial to make the standard solutions (3 individual STD vials to cover Mp 266-66000 Dalton) with concentration of 2.25 mg/mL for each standard. All standards are dissolved over 2 hr.

Note: Polystyrene standards and calibration curve shall be freshly prepared every time.

### Sample Preparation

For pure PLGA or PLA samples, weigh 10 mg of sample in a 1.5 mL Eppendorf tube. Add 1 mL of THF to dissolve polymer via an orbital shaker over 2 hr (room temperature). Centrifuge the dissolved polymer/THF sample at 14000 rpm for 2 minutes, take 100 μL of the supernatant for dilution to make the final 2 mg/mL sample for GPC analysis (100 μL of the supernatant+400 μL of THF).

For formulation samples, weigh a sufficient amount of formulation (corresponds to 10 mg of polymer) in a 1.5 mL Eppendorf tube. For example, for a 50% drug loading formulation with 50/50 PLGA to NMP ratio, 40 mg of the formulation shall be weighed. Centrifuge the dissolved formulation/THF sample at 14000 rpm for 2 minutes, take 100 μL of the supernatant for dilution to make the final 2 mg/mL sample for GPC analysis (100 μL of the supernatant+400 μL of THF).

### In Vitro Release of (+)-TBZ and (+)-(α)-DHTBZ from Injectable Suspensions

In vitro release was performed under sink condition for (+)-TBZ and (+)-(α)-DHTBZ suspension formulations. Volume of the release medium could be adjusted according to the depot size and drug loading (%) of the formulation. In one embodiment, 35 mg of a 30% drug loading formulation was injected into 400 mL pH 7.4 phosphate buffer saline with 0.2% (v/v) Tween 80 at 37° C. After solvent dissipation, an in-situ forming implant would form in the release medium. At predetermined time points, 0.5 mL of the release medium was withdrawn for HPLC analysis to calculate drug concentration in the release medium. The accumulated amount of drug released was calculated at predetermined time points to obtain the accumulated release profile.

### Size Reduction of (+)-TBZ and (+)-(α)-DHTBZ Particles via Jet Milling

Up to 5 grams of raw (+)-TBZ or (+)-(α)-DHTBZ powders were weighed and fed into the jet mill (Micromacinazione, Switzerland) at a rate of about 1 gram per 60 seconds. The feeding pressure and grinding pressure was tunable, dependent on the desired particle size to be collected. After milling (+)-TBZ or (+)-(α)-DHTBZ particles were collected, sealed, and preserved under the desired storage condition. In order to acquire large API particles (i.e. particles with D(50) above 100 μm) with narrow size distribution, jet milled API powders could also be further filtered through 25 μm filter using 0.5% w/w Tween 80 aqueous solution as the dispersant, followed by oven drying. Particle size was then measured by Malvern Mastersizer 3000 (Malvern Analytical Ltd, United Kingdom).

### Particle Size Analysis of (+)-TBZ and (+)-(α)-DHTBZ Particles

Particle size and size distribution of jet milled API particles were analyzed using Malvern Mastersizer 3000 using deionized water as the dispersing medium. Particle size distribution was measured and recorded. Table 1 categorized the three main particle size range in D(50) of the pharmaceutical active ingredient used in the following examples. This size categorized system would be adopted throughout the present application if not further described.

TABLE 1
Particle Size Range of (+)-TBZ and (+)-(α)-DHTBZ Particles

| Particle type | D(50) | |
|---|---|---|
| Small (S) | 10-35 | μm |
| Medium (M) | 50-80 | μm |
| Large (L) | 100-130 | μm |

### Polymer Molecular Weight Measurement

About 5-10 mg samples from each formulation was added into a 1.5 mL centrifuge tube and completely dissolved in 0.8 μL of THF. The solution was vortexed using a vortex shaker installed with a plate shaker until completely dissolved. Each sample was then centrifuged at 12,000 rpm for 2 minutes. The supernatant was collected and analyzed by GPC to determine the weight average molecular weight (Mw) and polydispersity index (PDI) of the polymer. Mw and PDI of a polymer were obtained by comparing with the polystyrene standards (Pskitr1L ReadyCal-Kit) with a Mp range from 266 to 66,000 Da. Table 2 summarized the Mw information of some the PLGA and PLA polymers being tested in the present application. Furthermore, polymer Mw change after gamma irradiation (25-40 kGy) was also measured and listed in Table 2.

TABLE 2
Molecular Weight of Biodegradable Polymers

| | | | Mw Change % |
|---|---|---|---|
| Type of Polymer | Mw | PDI | After irradiation |
| Resomer RG502 | 14046 | 1.7 | |
| Resomer RG503 | 32294 | 1.7 | |
| Resomer RG503H-TW | 30694 | 1.7 | |
| Resomer RG503H-US | 37549 | 1.6 | |
| PLGA 88-12 | 21342 | 1.7 | |
| PLA | 15598 | 1.9 | |
| irradiated* Resomer RG502 | 12339 | 1.9 | −17.8 |
| irradiated* Resomer RG503-TW | 26384 | 1.6 | −18.3 |
| irradiated* Resomer RG503H-TW | 22275 | 1.8 | −27.4 |
| irradiated* Resomer PLGA 88-12 | 16283 | 1.8 | −21.3 |

About up to 30% drop in polymer Mw was found for PLGA 5050 and 8812 after gamma irradiation at 25-40 kGy.

### Preparation of (+)-TBZ and (+)-(α)-DHTBZ Polymer Depot Compositions

(+)-TBZ and (+)-(α)-DHTBZ suspensions were prepared by filling weighed amount of API particles with desired particle size into a suitable luer-lock male syringe. A homogeneous polymer solution vehicle was prepared by mixing the weighed amount of polymer and biocompatible solvent(s) using a proper mixing device, i.e. a planetary mixer. Once prepared, a weighed amount of the polymer solution vehicle was filled into a suitable female, luer-lock syringe. Prior to injection, the male and female syringes were connected together, followed by back-and-forth mixing via the two plungers for up to 100 times to obtain uniform, milky or slightly yellowish suspensions. More preferably, the mixing was 75 times, and still more preferably, the mixing was 50 times. The final mixture for injection can be a viscous liquid, a gel, an emulsion, a suspension, or a semisolid dispersion, which is stable and ready for injection for preferably 30 minutes and more preferably stable and ready for injection within 1-2 hours without sedimentation and aggregation. Once the suspensions were ready, the female syringe was detached and a desired luer-lock needle was screwed onto the male syringe for injection. Preferably, a needle for injection was a 16-gauge needle, more preferably, an 18-gauge or 19-gauge needle, and most preferably, a 20-gauge or smaller size needle.

### Example 1. Formulation Uniformity

Formulation uniformity is crucial in developing any type of injectable dosage form. It ensures homogenous distribution of API in a drug product which enables consistent dosing every time. For pre-filled injectable dosage forms, it is also important to obtain consistent DL % with minimal variation among batches. In one embodiment, (+)-TBZ-polymer suspensions were prepared by a planetary mixer (MAZERUSTAR KK series Planetary Mixer, Kurabo Industries Ltd., Osaka, Japan) and then manually filled into 1 mL polypropylene (PP) syringe (Terumo, Japan) as pre-filled syringes that were ready for injection. Formulation uniformity was determined by examining the DL % of the formulations from three independently prepared batches with exactly the same compositions. DL % was measured by sampling the predetermined amount of formulation at the randomly selected section within the pre-filled syringes. Table 3 summarized the DL % results from three different RESOMER formulations. With less than 2% STD across different batches, it strongly indicates good formulation uniformity and minimal preparation variation which together deliver a promising potential for developing pre-filled (+)-TBZ-polymer formulations.

TABLE 3
Uniformity of (+)-TBZ-Polymer Depot Compositions

| | 1^{st} | 2^{nd} | 3^{rd} | |
|---|---|---|---|---|
| Formulation | Batch | Bach | Batch | Avg ± STD |
| 30% (+)-TBZ | 29.9% | 33.5% | 30.7% | 31.4 ± 1.9% |
| (S)-RG503/NMP 35/65 | | | | |
| 30% (+)-TBZ | 27.9% | 28.2% | 28.2% | 28.1 ± 0.2% |
| (S)-RG503H/NMP 35/65 | | | | |
| 30% (+)-TBZ | 30.4% | 29.1% | 30.4% | 30.0 ± 0.8% |
| (S)-RG502/NMP 45/55 | | | | |

S represents for (+)-TBZ particle size with D50 ranging from 10-35 μm.

In another embodiment, formulation uniformity of dual-syringe mixing was also demonstrated. A weighed amount of (+)-TBZ was filled in a suitable male, luer-lock PP syringe (for example, 1.2 mL male PP syringe from Qosina, USA), while a known amount of polymer solution vehicle was filled in a suitable female, luer-lock PP syringe (for example, 1.2 mL female PP syringe from Qosina, USA). The two syringes were then connected and mixed back-and-forth for 100 cycles to obtain the final suspension formulation that was ready for injection. Uniformity was determined by measuring the DL % at top, middle, and bottom section of a mixed syringe containing the final suspension. In addition, the physical stability of the suspension formulation was also investigated by again, measuring the DL % at top, middle, and bottom section of the syringe after 2 hours post dual syringe mixing. Table 4 summarizes the DL % analysis right after dual-syringe mixing and 2 hours post mixing. Both initial and 2-hr post mixing demonstrated minimal DL % difference across the entire length of the syringe (top, middle and bottom), indicating uniform mixing by dual syringes and good physical stability with no sedimentation within 2 hours after mixing, regardless to API particle size.

TABLE 4
Uniformity of Dual-Syringe Mixed Polymer Depot Compositions

| | | DL | DL | DL | |
|---|---|---|---|---|---|
| | | %: | %: | %: | |
| Formulation | | Top | Middle | Bottom | Avg ± STD |
| (+)-TBZ (S)- | Initial | 24.5 | 25.1 | 27.9 | 25.8 ± 1.8% |
| RG503/NMP 35/65 | 2-hr after | 24.1 | 25.8 | 27.0 | 25.6 ± 1.5% |
| (+)-TBZ (L)- | Initial | 23.2 | 25.7 | 28.8 | 25.9 ± 2.8% |
| RG503/NMP 40/60 | 2-hr after | 25.9 | 26.6 | 25.5 | 26.0 ± 0.6% |

S and L represent for (+)-TBZ particle size with D50 ranging from 10-35 and 100-130 μm, respectively.

In still another embodiment, (+)-(α)-DHTBZ suspensions formulation uniformity after dual-syringe mixing was also demonstrated. A weighed amount of (+)-(α)-DHTBZ was filled in a suitable male, luer-lock PP syringe (for example, 1.2 mL male PP syringe from Qosina, USA), while a known amount of polymer solution vehicle was filled in a suitable female, luer-lock PP syringe (for example, 1.2 mL female PP syringe from Qosina, USA). The two syringes were then connected and mixed back-and-forth for 100 times to obtain the final suspension formulation that was ready for injection. Uniformity was determined by measuring the formulation DL % at top, and bottom section of the syringe, as well as the injected portion via a 19G needle (Terumo, Japan). Table 5 summarizes the DL % analysis right after dual-syringe mixing. The DL % results demonstrated minimal difference across the syringe (top and bottom), as well as the injected portion through a 19G needle, indicating uniform mixing by dual syringes and good formulation injectability. In addition, (+)-(α)-DHTBZ particle size did not affect formulation uniformity after dual-syringe mixing. Good formulation uniformity after dual-syringe mixing was achieved, regardless small or large (+)-(α)-DHTBZ particles were tested in the formulations (Table 5).

TABLE 5
Uniformity of Dual-Syringe Mixed (+)-(α)-DHTBZ
Polymer Depot Compositions

| | | DL | DL | DL %: |
|---|---|---|---|---|
| | Theo. | %: | %: | Injected via |
| Formulation Composition | DL % | Top | Bottom | 19G needle |
| 40%(+)-(α)-DHTBZ | 40.0 | 40.5 | 39.7 | 39.5 |
| (L)-RG503/NMP 45/55 | | | | |
| 40%(+)-(α)-DHTBZ | 40.0 | 39.8 | 40.2 | 40.1 |
| (L)-RG503/NMP 50/50 | | | | |
| 40%(+)-(α)-DHTBZ | 40.1 | 39.7 | 38 | 39.4 |
| (S)-RG503/NMP 50/50 | | | | |
| 40%(+)-(α)-DHTBZ | 39.6 | 38.6 | 39.5 | 37.2 |
| (L)-RG503/NMP 50/50 | | | | |

S and L represent for (+)-TBZ particle size with D50 ranging from 10-35 and 100-130 μm, respectively.

### Example 2. Sustained Release of (+)-TBZ and (+)-(α)-DHTBZ from Polymeric Formulations

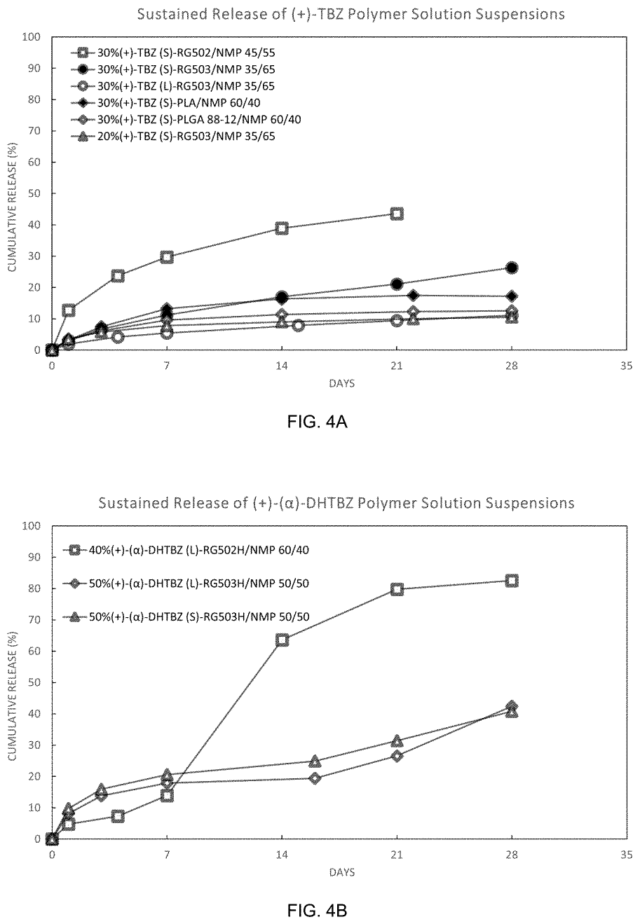

In one embodiment, at 30% drug loading with small (+)-TBZ particles (D(50): 10-35 μm), a formulation composed of RG502 and NMP at 45/55 w/w ratio showed sustained release with about 12.5% initial burst, followed by about 30% and 45% in vitro release at 1 and 3-week (FIG. 4A), respectively. In another embodiment, we enabled the methods of suppressing burst release of VMAT2 inhibitors by using varied combinations of RG503 and NMP. At the same 30% drug loading with small (+)-TBZ particles, formulations composed of RG503 and NMP at 35/65 w/w ratio demonstrated slower in vitro release, with <5% burst release at first 24h and only about 20% release after 3-week (FIG. 4A). In still another embodiment, we demonstrated that initial burst could still be kept below 4% when drug loading was raised up to 50% while 3-week release was still less than 30% [formulation composed of 50% (+)-TBZ (S)-RG503/NMP at 35/65 w/w ratio, FIG. 4A]. In another example, we demonstrated that release profiles were the same at drug loading between 20 or 30% for formulations composed of RG503/NMP at 35/65 w/w ratio (FIG. 4A), which was also unexpected since a 10% drug loading increase in a PLGA in situ forming depot drug delivery system would generally result in higher initial burst and faster overall release. Such enabling of increased drug loading without altering release profile is beneficial to develop a formulation with reduced injection volume.

On the other hand, PLA is a polymer made up of more hydrophobic lactic acid units, which requires longer time to degrade and typically is used for long-term sustained release injectables (4-6 months, in general), compared to poly (glycolic acid) (PGA) (Blasi et al., Journal of Pharmaceutical Investigation, 2019 (49) pg. 337-346). The same is true for PLGA polymers. Generally speaking, PLGA polymers with higher lactide content (more hydrophobic) would require longer time to degrade. Therefore, in still some other embodiments, polymer solution vehicles composed of PLA, or PLGA 88/12 with NMP were adopted to prepare (+)-TBZ suspensions. In comparing PLA and PLGA (RG502) with comparable molecular weight (about 15000 Da), longer lasting PLA indeed displayed slower in vitro release profile (FIG. 4A). PLGA 88-12/NMP 60/40 formulations at 30% drug loading demonstrated even slower in vitro release, with only about 15% 3-week release. Thus, in the present application, we enabled tunable polymer-based delivery systems that demonstrated varied duration for the delivery of (+)-TBZ with low or no initial burst.

In the present application, we also enabled sustained delivery of (+)-(α)-DHTBZ from formulations composed of biodegradable, polymeric vehicles. In one example (FIG. 4B), a formulation composed of 40% (+)-(α)-DHTBZ (L)-RG502H/NMP at 60/40 w/w ratio demonstrated drug release in a sustained manner with <5% initial burst release and about 80% accumulated release over a period of 3 weeks. On the other hand, we demonstrated tunable release profiles using (+)-(α)-DHTBZ as the active pharmaceutical ingredient (API) in polymer depot compositions with low initial burst release, yet high drug loading. In one example, a formulation composed of 50% (+)-(α)-DHTBZ (L)-RG503H/NMP at 50/50 w/w ratio displayed only about 40% accumulated release over 4 weeks in vitro (FIG. 4B).

### Example 3. Effect of Polymer/Solvent Ratio on Release of (+)-TBZ and (+)-(α)-DHTBZ

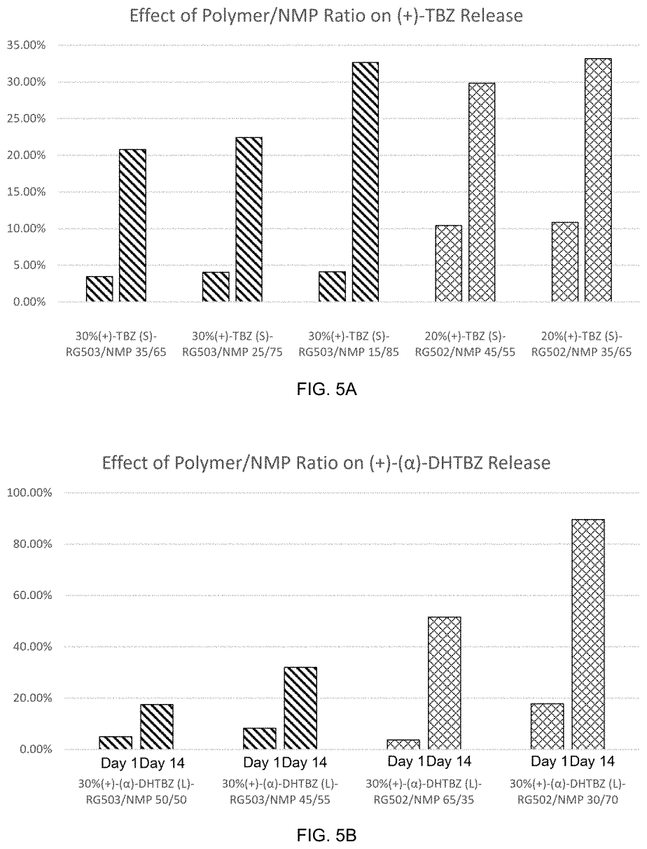

It is well-acknowledged that the content of polymer (%) used in a formulation would have a notable impact on drug release. Generally speaking, the higher the content of polymer (%), the slower drug release. The polymer solution becomes more viscous as the content of polymer (%) increases, which leads to a lower burst and slower drug release. In addition, the higher content of the polymer, the longer time it requires for API to diffuse out of the depot matrix, which also leads to a slower drug release. It is demonstrated herein that in vitro release of (+)-TBZ and (+)-(α)-DHTBZ from polymer formulations can be tailored by adjusting the polymer to solvent ratio. Understanding how influential changing polymer/solvent ratio on release can be very important not only in the fine-tuning for subsequent formulation development, but also in identifying the tolerability of production variation. To explore candidates for long-acting, sustained delivery of VMAT2 inhibitors preferably for 1-month, and more preferably for 2-month duration, RG503/NMP and RG502/NMP formulations were further investigated. FIG. 5A shows the effect of polymer/NMP ratio on in vitro release of (+)-TBZ. Unexpectedly, polymer/NMP ratio has limited or no effect on initial burst release of (+)-TBZ, with <5% and about 10% release for RG503/NMP (30% drug loading) and RG502/NMP (20% drug loading) formulation, respectively (FIG. 5A). This was quite unique since lower polymer concentration (%) would generally result in higher initial burst in PLGA in-situ forming depot dosage forms. In this case, for RG503/NMP formulations at 30% drug loading, whether polymer% was 35%, or 25%, or 15%, no initial burst was found. However, at 30% drug loading, faster overall release was observed when RG503% decreased from 35% w/w to 15% w/w in the polymer solution vehicles (FIG. 5A). The same trend was also observed in formulations at 20% drug loading with the small API particles using RG502 with 45/55 and 35/65 w/w polymer/NMP ratio. Notably, the influence of polymer/NMP ratio was not significant for the first few days and this allows one to adjust overall drug release without altering initial burst release, which is typically regarded as the key to determine a suitable in vivo peak to trough (P/T) for controlled drug delivery. Still worth noting, at 30% drug loading the release profiles of RG503/NMP at 35/65 and 25/75 w/w ratio are considerably similar. This particular finding may merit the manufacturing process and downstream product development, since a wider polymer/NMP range can be accepted for the desired release profile.

On the other hand, to examine whether this characteristic release profile affected by adjusting the polymer/solvent ratio can be reproduced with another VMAT2 inhibitor, we conducted the same release studies with (+)-(α)-DHTBZ which was just a reduced form of (+)-TBZ that share very much similar chemical structure (FIG. 5B). Unlike (+)-TBZ whose initial burst was almost not affected by polymer/solvent ratio, for (+)-(α)-DHTBZ PLGA polymer suspensions, lower content of polymer (%) resulted in not only overall faster release but also higher initial burst in both cases of RG502/NMP and RG503/NMP polymer solution vehicles (30% drug loading). With such a small structural difference, these two VMAT2 inhibitors still demonstrated significantly different patterns of release profiles in correspondence to their relative polymer% in the formulations. It is obviously rather difficult for a person having ordinary skill in the art to predict release characteristics simply by following prior arts. Furthermore, formulations composed of lower content of polymer (%) that showed higher burst release can be utilized as the “boost” dose in a regimen to bring up plasma (+)-(α)-DHTBZ level at the early stage to assure VMAT2 inhibitor in the therapeutic range.

### Example 4. Effect of Hydrophobic Solvents on In Vitro Release

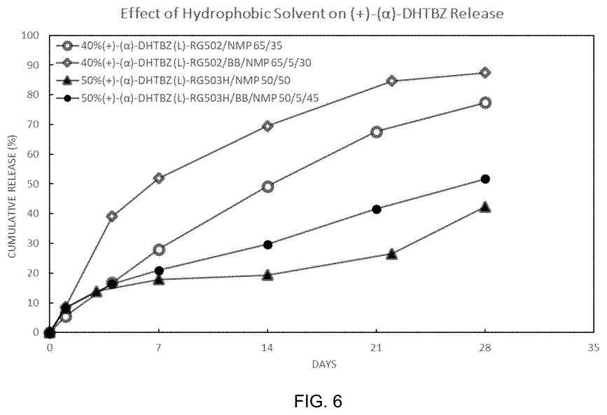

In situ forming depot drug delivery system has become a prevailing approach for parenteral applications owing to the advantages of: biodegradable/biocompatible, high drug loading, better patient compliance, and reduced administration frequency. However, it is extremely challenging to achieve zero-order release profile in long-term delivery systems, typically due to the issue of initial burst, caused by fast dissipation of hydrophilic solvent into body fluid. One potential approach to avoid initial burst is to introduce hydrophobic solvent(s) into the PLGA polymer solution vehicle to slow-down solvent diffusion so as to prolong drug release. BB, BA, and triacetin are some of the commonly available, biocompatible, hydrophobic solvents that have been tailored with NMP to control drug release in ATRIGEL® drug delivery systems and the like. Surprisingly, we found BB was able to alter the release of (+)-(α)-DHTBZ in an opposite manner as one skill in the art would expect (FIG. 6). In one embodiment, a formulation composed of 40% (+)-(α)-DHTBZ (L)-RG502/BB/NMP at 65/5/30 ratio demonstrated substantially faster release when only 5% (w/w) of the NMP in the polymer solution vehicle was substituted with BB (14-day release increased from about 50% to about 70%). In another embodiment, a formulation composed of 50% (+)-(α)-DHTBZ (L)-RG503H/BB/NMP at 50/5/45 ratio also demonstrated accelerated release after replacing 5% (w/w) of NMP in the polymer solution vehicle by BB (14-day release increase from about 20% to about 30%). We also found replacement of NMP by this small portion of BB only led to faster accumulated release but actually did not affect the initial release, which was also unexpected since typically introducing BB would increase polymer solution viscosity and help reduce the initial burst. These findings were exceptional to what the prior arts in the related field have revealed. Furthermore, such method used herein provides an advantage on controlling overall release profile of VMAT2 inhibitors without affecting initial release which can potentially help keep low plasma P/T ratio in a living subject. This is very difficult to achieve when developing a sustained-release, in situ forming depot drug delivery system.

### Example 5. Effect of Drug Loading on In Vitro Release

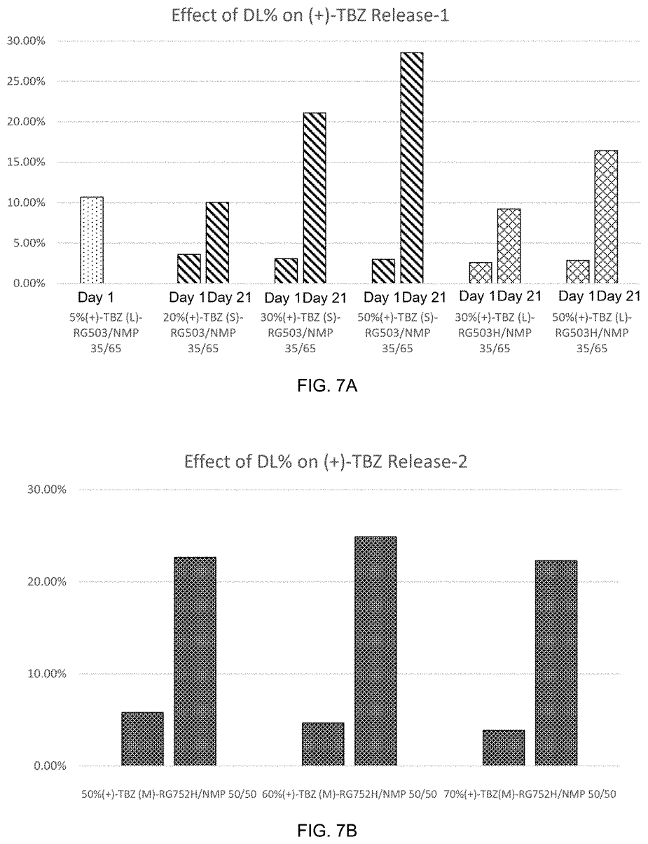

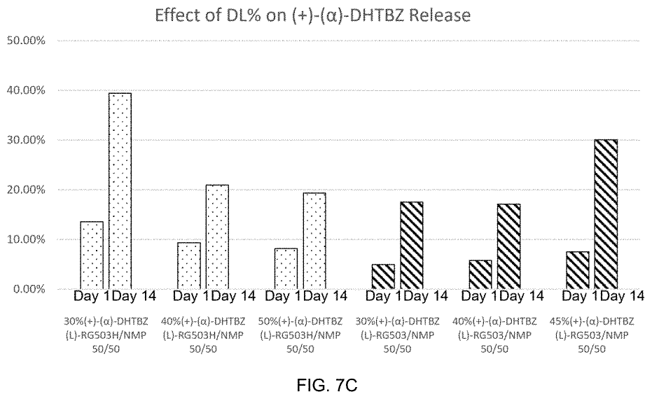

Drug loading (DL %) in an in-situ forming, injectable sustained release depot/implant formulation is highly critical for that it determines dosing volume and how long the therapeutic effect can last. Typically, the lower the injection volume, the better the patient compliance, since with reduced injection time required, the less pain a patient would suffer. Because the formulation composed of 30% (+)-TBZ (S)-RG503/NMP at 35/65 w/w ratio demonstrated slow and sustained in vitro release, we further explored the DL % effect on the drug release profile from this identical polymer solution vehicle. Tailored release profiles of (+)-TBZ are obtained by adjusting the DL % in a vehicle composed of RG503/NMP at 35/65 ratio. In one embodiment, increasing the DL % to 50% resulted in significantly faster release of (+)-TBZ, compared to the formulation composed of the same vehicle but at 30% drug loading (FIG. 7A). Unexpectedly, the initial burst was not affected by the increased DL % from 20 to 50%, but only the release rate afterward. This would provide a beneficial tuning approach for developing long-term sustained release in-situ forming depot/implant formulations, due to the fact that under certain circumstances, a faster overall release profile without high initial burst is preferred to avoid large plasma level fluctuation (smaller plasma level P/T ratio).

To understand whether DL % affects release in a similar way on other biodegradable polymers, we further investigated formulations composed of (+)-TBZ (L)-RG503H/NMP at 35/65 w/w ratio with varied DL % and found the identical trend. While initial burst remained the same, increasing DL to 50% provided faster in vitro release profile than 30% drug loading (FIG. 7A). Such a trend remained true regardless of whether small or large API particles were tested. These results further strengthen the approach of using DL % as a tool to adjust release profile while effectively avoiding elevated initial burst.

More surprisingly, in another embodiment, we demonstrated that both initial release and overall release profile were not noticeably affected by increasing DL % from 50% to 60% and to 70% in formulations composed of (+)-TBZ (M)-RG752H/NMP at 50/50 w/w ratio (FIG. 7B). This would benefit formulation design in a different way, which enables the development of sustained release VMAT2 inhibitor in-situ forming depot with reduced dosing volume, while maintaining a low initial burst and a similar release profile while the DL % is raised from 50% to 60% or 70%.

In another embodiment, the DL % effect of (+)-(α)-DHTBZ on release profile was investigated in polymer solution or suspension formulations. Similar to what were found from (+)-TBZ, the DL % affected (+)-(α)-DHTBZ release on the initial burst and the overall release rate differently. While the initial burst release only changed slightly with varying the DL %, the overall release rate was faster when (+)-(α)-DHTBZ loading was increased from 30% to 45% in the vehicle composed of RG503/NMP at 50/50 w/w ratio. However, it was unique that release profiles were exceptionally almost identical at 30 and 40% drug loading (FIG. 7C). This was quite advantageous as it is common to develop sustained-release drug products in varied dose strengths, and a good dose proportionality could be achieved without increased initial burst for the formulations with increased dose strength. What is more unexpected, varying the DL % worked completely opposite to carboxylic acid-terminated RG503H and (+)-(α)-DHTBZ. The release of (+)-(α)-DHTBZ was indeed slower with increasing DL % in the RG503H formulations (FIG. 7C). Such findings once again, re-emphasize that the effect of drug loading on release profiles of VMAT2 inhibitors cannot be reproduced by simply mimicking or reproducing formulations in other related prior arts disclosed elsewhere.

### Example 6. Effect of (+)-TBZ and (+)-(α)-DHTBZ Particle Size on Release from Polymeric Formulation

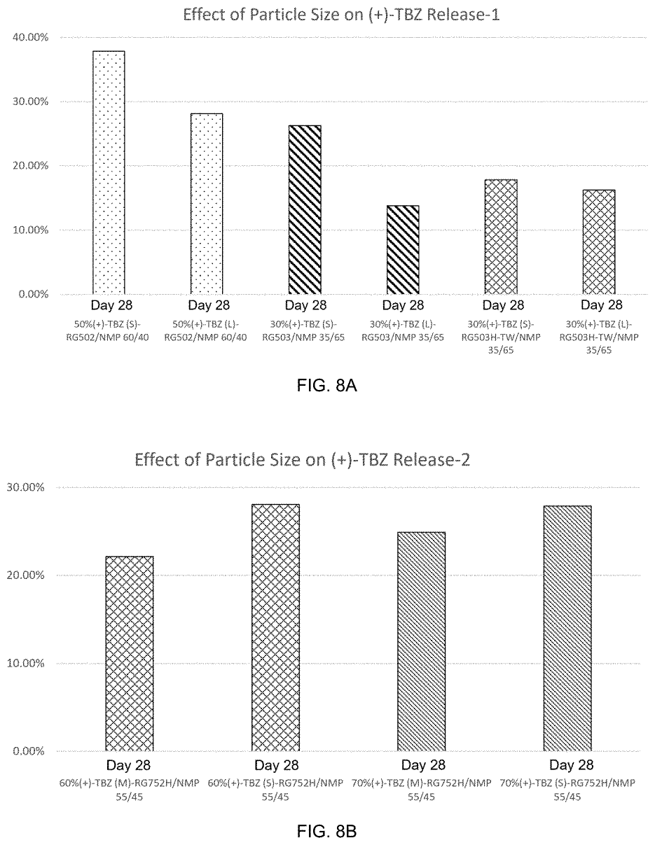

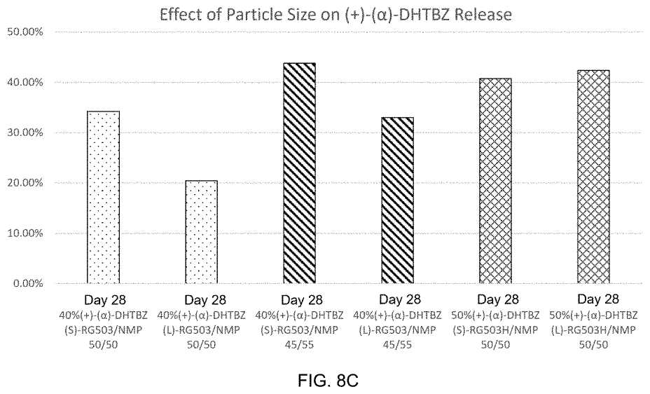

Typically speaking, particle size shall alter release profile in suspension formulations (Drug Des. Devel. Ther. 2013; 7: 1027-1033). Dissolution rate is positively correlative to surface area of the particles in a suspension formulation. While specific surface area increases with decreasing particle size of the API particles, so does the drug dissolution rate. A substantial difference in dissolution rate can exist according to the variation on particle size and the relative surface area, especially during the initial period of the dissolution study. FIG. 8A demonstrated the adjustment of (+)-TBZ release in RESOMER/NMP polymer solution formulation using (+)-TBZ with different particle sizes. For both polymer solution vehicles composed of RG503/NMP at 35/65 w/w ratio and RG502/NMP at 60/40 w/w ratio, large API particles (D(50): 100-130 μm) indeed displayed slower in vitro release than small particles did, regardless of DL % was at 30 or 50%.

However, unexpectedly, we found that such particle size effect on (+)TBZ release may be specific only in combination with certain types of polymer solution vehicles. As demonstrated in FIG. 8A, drug release from suspension formulations consist of (+)-TBZ particle and RG503H/NMP solution was unexpectedly not affected by API particle sizes. In a similar manner, particle size of (+)-TBZ showed limited effect on release from polymer solution vehicles composed of RG752H/NMP at 55/45 w/w ratio, regardless DL % was at 60% or 70% (FIG. 8B). This was even more surprising because PERSERIS® (a commercially available product composed of risperidone suspended in PLGA polymer with the same carboxylic acidic terminal function group, PLGH 8020) revealed that there is a trend of increased release rate with decreased API particle size (PERSERIS® FDA Product Quality Review). Such unique finding in the present application suggested that sustained release of (+)-TBZ from polymeric formulation cannot be easily achieved by simple mimicking formulations disclosed in the related prior arts.

In another approach, polymer solution-based suspensions composed of (+)-(α)-DHTBZ as API were also being investigated for long-term, sustained release of VMAT2 inhibitors for the treatment of TD. In one embodiment, a formulation composed of RG503/NMP at 50/50 w/w ratio and small (+)-(α)-DHTBZ particles at 40% drug loading demonstrated overall faster in vitro release, compared to the formulation composed of large (+)-(α)-DHTBZ particles but exactly identical polymer solution vehicle (FIG. 8C). Such tendency remained true among polymer solutions composed of RG503/NMP at various solvent ratios (i.e. 50/50 and 45/55, FIG. 8C). It is worth to note that similar to a lack of size effect of (+)-TBZ on release from RG503H (acid terminated RG503), the particle size of (+)-(α)-DHTBZ indeed also showed no impact on release rate from the same carboxylic acid-terminated polymer solution vehicle. It is clear that there is discrepancy in how API particle size can influence drug release in different polymer solution vehicles. While API particle size did demonstrate its effect on drug release profile in some of the polymer solution-based formulations, the release profile obviously did not echo with API particle size variation in certain types of polymer solution vehicles, for example RG503H or RG752H. Once again, such unique findings in the present application suggested that sustained release of VMAT2 inhibitors from a polymeric formulation cannot be achieved by simply mimicking or reproducing formulations in other related prior arts disclosed elsewhere.

### Example 7. Effect of Terminal Sterilization on In Vitro (+)-(α)-DHTBZ Release

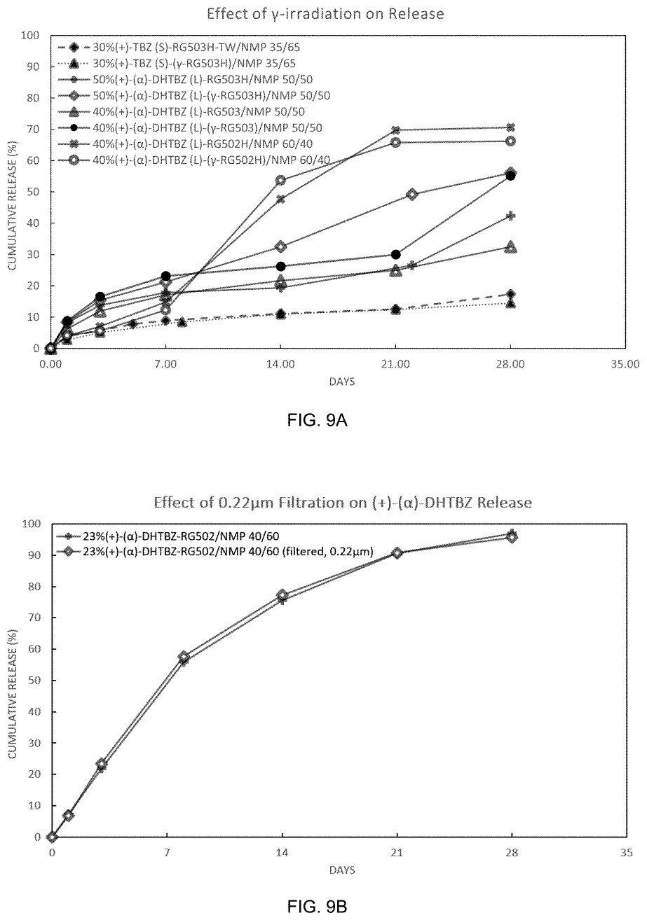

Gamma irradiation is an effective terminal sterilization method for injectable products as well as medical devices in that it can be performed typically under ambient conditions and is of high energy penetration capability (change of packaging is generally not required). However, for sustained-release, biodegradable, polymer solution vehicle-based formulation, gamma irradiation may be a big hurdle due to the fact that polymer degradation can occur during such sterilization process, or, polymer stability may be vulnerable post-gamma irradiation. Furthermore, it is generally acknowledged that polymer Mw can lead to different vehicle viscosity, varied degradation rate, and solidification speed upon contact with the aqueous medium, which certainly would cause a dramatic impact on the release profile of a formulation. It is generally believed that higher Mw polymers would generally solidify faster than the lower Mw polymers, thus results in decreased initial burst (Eliaz et al., Journal of Biomedical Materials Research, 50 (3), 2000). In addition, for formulations comprised of PLGA polymer/NMP with 50/50 lactide to glycolide ratio, the one made of smaller Mw PLGA polymer (RG 502H) formed an implant with higher porosity and larger pores, compared to the one made of larger Mw polymer, RG 504H, thus demonstrated increased initial burst (Asaneh et al., Journal of Pharmaceutical Sciences, 98 (1), 2009). On the other hand, initial release can also be affected by vehicle viscosity; for example, a formulation composed of polymer with smaller Mw is less viscous than that composed of polymer with larger Mw, which would, on the contrary, cause faster solvent dissipation and resulting in a higher initial burst. In a more complicated manner, Mw difference is indicative of different polymer chain length which would also determine the time required for the polymers to degrade and thus change the drug release rate. All in all, polymer Mw are recognized to have significant impact on sustained drug release. One would, therefore, expect any cause that can make polymer Mw varied would change drug release results. In one embodiment, we thus evaluated the impact of gamma sterilization on RG 503H polymer. Polymer was first gamma irradiated at about 35 kGy then made into (+)-TBZ-RG503H/NMP formulation. Surprisingly, although polymer Mw dropped nearly 30% from 30,692 to 22,275 (MW, Table 2) after gamma irradiation at 35 kGy, we did not find changes on in vitro release profile of the formulation composed of 30% (+)-TBZ (S)-RG503H/NMP at 35/65 w/w ratio (FIG. 9A) up to 28 days. This consistent release profile, independent of Mw change, unexpectedly, appeared that gamma irradiation can be a potential terminal sterilization method for such a formulation. On the other hand, in another embodiment, gamma sterilization (25-30 kGy) was invested for some viscous (+)-(α)-DHTBZ polymer suspensions. Unexpectedly, while accelerated release was found from post-irradiation formulations composed of 40% (+)-(α)-DHTBZ (L)-RG503/NMP at 50/50 w/w ratio and composed of 50% (+)-(α)-DHTBZ (L)-RG503H/NMP at 50/50 w/w ratio, the release profiles for a formulation composed of 40% (+)-(α)-DHTBZ (L)-RG502H/NMP at 60/40 w/w ratio almost did not change after the gamma irradiation (FIG. 9A).

Alternative sterilization approaches that are not detrimental to polymer are also highly sought after. For example, filtration through 0.22 μm filter could be another option for terminal sterilization. Nonetheless, in order to provide long-term release in a sustained manner, PLGA or PLA-based formulations generally come in as viscous solutions or suspensions, which make filtration very problematic. In one embodiment, (+)-(α)-DHTBZ polymer suspensions made of RG502/NMP at 40/60 w/w ratio was prepared at 23% drug loading. Filtration of such vehicle through a 0.22 μm disc filter was easy and straightforward. In vitro release profile of the formulations made of filtered and non-filtered polymer solution vehicles were identical (FIG. 9B), which demonstrated the feasibility of using 0.22 μm filtration as the terminal sterilization process for those less viscous polymer solutions-based formulations.

In the present application, we demonstrated that either 0.22 μm filtration or gamma irradiation could be applied as terminal sterilization process for the proposed polymer depot compositions containing VMAT2 inhibitors for TD treatment.

### Example 8. PK of Subcutaneous Administrated (+)-TBZ-RG503/NMP 35/65 Suspensions in Rats

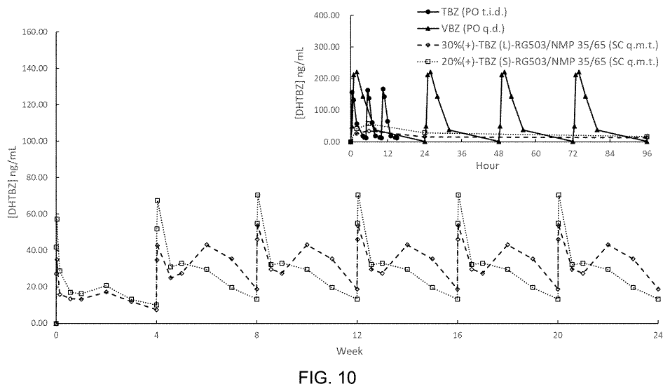

In one example, a PK study of formulations composed of (+)-TBZ-RG503/NMP at 35/65 w/w polymer ratio was conducted with Sprague Dawley (SD) rats. These formulations were selected for their sustained in vitro release without high initial burst as demonstrated in the earlier examples in the present application. Polymer solution or suspension formulations containing (+)-TBZ were prepared as previously described. In one embodiment, formulations composed of 30% (+)-TBZ (L)-RG503/NMP at 35/65 w/w ratio and 20% (+)-TBZ (S)-RG503/NMP at 35/65 w/w ratio were subcutaneously administrated to SD rats (N=3) at a dose level of 60 mg/kg, while others received TBZ or VBZ solution at a dose level of 10 mg/kg via oral gavage (N=3) as references. The animals that received formulations containing (+)-TBZ were dosed on Day 1, followed by blood sampling at 2, 6, 12, 24 hours and 4, 7, 14, 21, 28, 35, 42, 49, 56 days post-dosing. For the animals that received orally given TBZ or VBZ suspensions, blood sampling was performed at 2, 6, 12, 24, and 48 hours post-dosing. For each animal, both plasma (+)-(α)-DHTBZ and (+)-TBZ concentrations were measured via LC-MS. PK results were evaluated by the sum of plasma (+)-(α)-DHTBZ and (+)-TBZ level together. Furthermore, PK simulation for monthly repeated dosing of the two (+)-TBZ-RESOMER suspensions as well as the daily repeated dosing of TBZ or VBZ were obtained and presented as FIG. 10. The lower large diagram of FIG. 10 demonstrated that both (+)-TBZ-polymer suspensions were able to successfully provide sustained release of VMAT2 inhibitors ((+)-(α)-DHTBZ and (+)-TBZ) for one month, with small P/T ratio. In addition, throughout the PK study, plasma levels of VMAT2 inhibitors released from the polymeric formulations fall in between a window of 10-200 ng/mL, which is in the range of plasma level given by the oral dosage form of VBZ. More significantly, the present application enabled the polymer depot compositions which can provide sustained release of VMAT2 inhibitors with significantly less plasma level fluctuation, comparing to other commercially available drug products. XENAZINE® (TBZ, racemic mixtures of (+) and (−)-TBZ) is designed to be given three times a day for the treatment of TD, while Ingrezza (VBZ) is prescribed for once daily administration. PK simulation of thrice-a-day repeated dosing of TBZ and once-a-day repeated dosing of VBZ were compared side-by-side with PK simulations of monthly repeated dosing of formulations composed of 30% (+)-TBZ (L)-RG503/NMP at 35/65 w/w ratio and 20% (+)-TBZ (S)-RG503/NMP at 35/65 w/w ratio (Top, small diagram of FIG. 10). While daily delivery of TBZ or VBZ [for VBZ group, plasma level only demonstrated as (+)-(α)-DHTBZ] showed obviously large plasma fluctuation of the VMAT2 inhibitors, both (+)-TBZ-polymer depot compositions enabled substantially smaller P/T ratio of VMAT2 inhibitors. The formulations presented in this application undoubtedly posed great potential to be used as sustained release medications requiring much lower dosing frequency, yet are able to continuously deliver VMAT2 inhibitors within effective therapeutic ranges, which shall significantly reduce side effects that accompany the currently available treatment of XENAZINE®.

### Example 9. PK of Subcutaneous Administrated (+)-TBZ-RG752H/NMP 65/35 and (+)-TBZ-RG503/NMP Polymer Depot Compositions in Rats

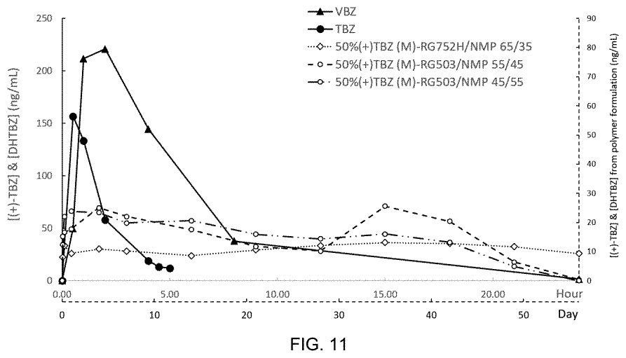

After demonstrating the feasibility of using polymer solution vehicles composed of RG503 for 1-month delivery of VMAT2 inhibitors, we further explored on extending dosing duration by two approaches: 1. replacing RG503 with other PLGA polymers, but of higher lactide to glycolide ratio; and 2. Using the same RG503 polymer, yet with raised polymer to NMP ratio. Furthermore, to avoid large injection volume, we also investigated formulations with higher DL % (>40%) to achieve the same low P/T ratio, sustained delivery of VMAT2 inhibitor via polymer solution formulations. In one embodiment, formulations comprised of 50% (+)-TBZ (M)-RG752H/NMP at 65/35 w/w ratio, 50% (+)-TBZ (M)-RG503/NMP at 55/45 w/w ratio and 50% (+)-TBZ (M)-RG503/NMP at 45/55 w/w ratio were prepared in the same method as previously described. In-situ forming implants at dose level of 50 mg/kg were subcutaneously administrated to SD rats (N=3), followed by blood sampling at 2, 6, 12, 24 hours and 4, 7, 14, 21, 28, 35, 42, 49, 56, and 60 days post-dosing. As references, TBZ and VBZ suspensions at dose level of 10 mg/kg were given to SD rats via oral gavage, followed by blood sampling at 2, 6, 12, 24 and 48 hours (N=3). PK results are shown in FIG. 11 as a double-axes chart. The solid axes correspond to plasma (+)-TBZ and (+)-(α)-DHTBZ level over time for the animals received orally given TBZ or VBZ [for VBZ group, plasma level only demonstrated as (+)-(α)-DHTBZ] suspensions, while the dashed axes correspond to plasma (+)-TBZ and (+)-(α)-DHTBZ levels over time for the animals that received subcutaneously injected polymer solution formulations. Encouragingly, while large plasma level fluctuation was observed for animals that received TBZ or VBZ suspensions orally, significantly less VMAT2 inhibitors plasma level fluctuation was found for the animals that received formulations comprised of 50% (+)-TBZ (M)-RG752H/NMP at 65/35 w/w ratio, 50% (+)-TBZ (M)-RG503/NMP at 55/45 w/w ratio and 50% (+)-TBZ (M)-RG503/NMP at 45/55 w/w ratio. What we demonstrated in this application was of valuable advantages for TD treatment, since dose titration may be unnecessary for such long-termed, subcutaneously delivered dosage forms with such small plasma P/T ratio, provided that plasma level of VMAT2 can be maintained and held in the desired range. Moreover, the duration of two formulations comprised of 50% (+)-TBZ (M)-RG503/NMP at 55/45 w/w ratio and 50% (+)-TBZ (M)-RG503/NMP at 45/55 w/w ratio were able to maintain (+)-TBZ and (+)-(α)-DHTBZ plasma level in rats (which was within the therapeutic window for rats administrated with VBZ via oral route) for at least one and a half months. More encouragingly, the formulation composed of 50% (+)-TBZ (M)-RG752H/NMP at 65/35 w/w ratio successfully enabled even longer duration. Rat plasma level of (+)-TBZ and (+)-(α)-DHTBZ together was above 10 ng/mL over 60-day PK study, which P/T ratio was less than 2 (FIG. 11). Last but not least, with raised DL % to 50% w/w, injection volume could be significantly reduced, compared to DL % at 30% or less. Nevertheless, such finding was unique since in this application we enabled high drug loading polymer formulations without initial burst release. A long-term, sustained release formulation with a smaller injection volume would most definitely enhance patient compliance.

### Example 10. PK of Subcutaneously Administrated (+)-TBZ-PLGA 88-12/NMP Formulations in Rats

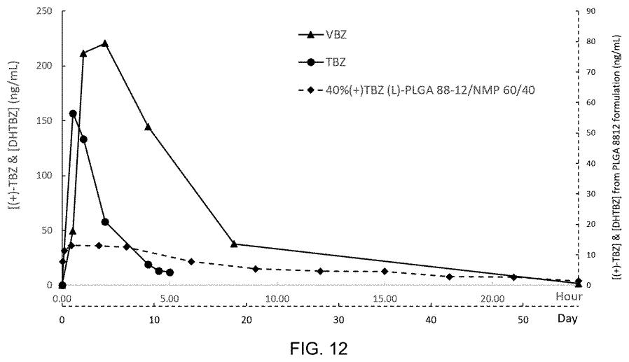

PLA is a polymer made up of small lactic acid units, which takes longer time to degrade, compared to PGA. After demonstrating the feasibility of using polymer solution vehicles composed of PLGA at 50/50 ratio for 1-month delivery of VMAT2 inhibitors, we further explored to extend delivery duration by replacing PLGA 50/50 with other PLGA polymers having higher lactide to glycolide ratios. In one embodiment, a formulation comprised of 40% (+)-TBZ (L) was prepared in the same method as previously described, but using PLGA 88-12/NMP at 60/40 w/w ratio as the polymer solution vehicle. In-situ forming implants at dose level of 60 mg/kg was subcutaneously administrated to SD rats (N=3), followed by blood sampling at 2, 6, 12, 24 hours and 4, 7, 14, 21, 28, 35, 42, 49, 56 days post-dosing. As references, TBZ and VBZ suspensions at dose level of 10 mg/kg were given to SD rats via oral gavage, followed by blood sampling at 2, 6, 12, 24 and 48 hours (N=3). PK results are shown in FIG. 12 as a double-axes chart. The solid axes correspond to plasma (+)-TBZ and (+)-(α)-DHTBZ level over time for the animals that received orally given TBZ or VBZ [for VBZ group, plasma level only demonstrated as (+)-(α)-DHTBZ] suspensions, while the dashed axes correspond to plasma (+)-TBZ and (+)-(α)-DHTBZ levels over time for the animals that received subcutaneously injected 40% (+)-TBZ-PLGA 88-12/NMP 60/40 suspensions. Surprisingly, while a large plasma level fluctuation was observed for animals that received TBZ or VBZ suspensions orally, significantly less VMAT2 inhibitors plasma level fluctuation was found for the animals that received 40% (+)-TBZ (L)-PLGA 88-12/NMP 60/40 suspension. This is of considerable advantage for TD treatment, since dose titration may be unnecessary for such long-termed, subcutaneously delivered dosage forms with small plasma P/T ratio, provided that the plasma level of VMAT2 can be maintained and held in the desired range. Furthermore, the duration of 40% (+)-TBZ (L)-PLGA 88-12/NMP 60/40 suspension were able to release (+)-TBZ and (+)-alpha-DHTBZ for at least 2 months.

### Example 11. PK of Subcutaneously Administrated (+)-(α)-DHTBZ-PLGA 50-50/NMP Formulations in Rats

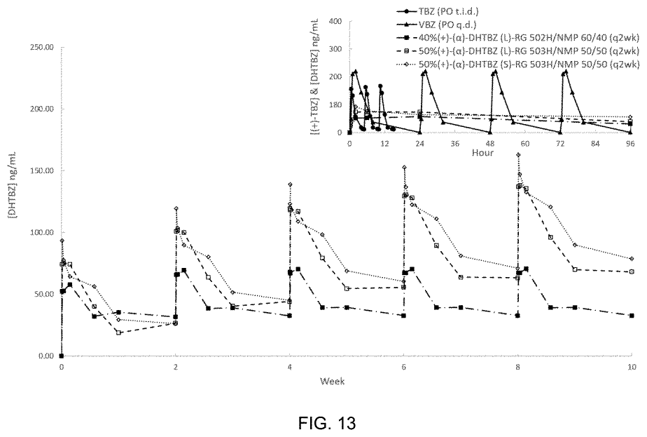

In vivo animal studies were conducted with SD rats using formulations composed of 40% (+)-(α)-DHTBZ (L)-RG502H/NMP at 60/40 w/w ratio, 50% (+)-(α)-DHTBZ (L)-RG503H/NMP at 50/50 w/w ratio, and 50% (+)-(α)-DHTBZ (S)-RG503/NMP at 50/50 w/w ratio. All suspensions were selected for their sustained in vitro release without high initial burst as demonstrated in the earlier examples in this application. (+)-(α)-DHTBZ polymer depot compositions were prepared as previously described. In one embodiment, formulations composed of: 40% (+)-(α)-DHTBZ (L)-RG502H/NMP at 60/40 w/w ratio, 50% (+)-(α)-DHTBZ (L)-RG503H/NMP at 50/50 w/w ratio, and 50% (+)-(α)-DHTBZ (S)-RG503H/NMP at 50/50 w/w ratio were subcutaneously administrated to SD rats (N=3) at a dose level of 50 mg/kg, while others received TBZ or VBZ suspensions at a dose level of 10 mg/kg via oral gavage (N=3) as references. The animals received (+)-(α)-DHTBZ-polymer suspensions were dosed on Day 1, followed by blood sampling at 2, 6, 12, 24 hours and 4, 7, 14, 21, 28, 35, 42, 49, 56 days post-dosing. For the animals that received orally given TBZ or VBZ suspensions, blood sampling was performed at 2, 6, 12, 24, and 48 hours post-dosing. For each animal, plasma (+)-(α)-DHTBZ concentrations were measured via LC-MS to evaluate the 35-Day PK profiles. Furthermore, PK simulation for bi-weekly repeated dosing of the (+)-(α)-DHTBZ-RESOMER suspensions as well as the daily repeated dosing of TBZ or VBZ were obtained and presented as FIG. 13. The lower large diagram of FIG. 13 demonstrated that all (+)-(α)-DHTBZ-polymer suspensions were able to successfully provide sustained release of VMAT2 inhibitors for 2 weeks, with a low P/T ratio. In addition, throughout the PK study, plasma levels of VMAT2 inhibitors released from the polymeric formulations fall in between a window of 10-200 ng/mL, which is in the range of the plasma levels given by the oral dosage form of VBZ. More significantly, the present application enabled the polymer depot compositions which can provide sustained release of VMAT2 inhibitors with significantly less plasma level fluctuation, compared to other commercially available drug products. XENAZINE® (TBZ, racemic mixtures of (+) and (−)-TBZ) is designed to given three times a day for the treatment of tardive dyskinesia (TD), while Ingrezza (VBZ) is prescribed for once daily administration. PK simulation of thrice-a-day repeated dosing of TBZ and once-a-day repeated dosing of VBZ were compared side-by-side with PK simulations of bi-weekly repeated dosing of formulations composed of 40% (+)-(α)-DHTBZ (L)-RG502H/NMP at 60/40 w/w ratio, 50% (+)-(α)-DHTBZ (L)-RG503H/NMP at 50/50 w/w ratio, and 50% (+)-(α)-DHTBZ (S)-RG503H/NMP at 50/50 w/w ratio (Top, small diagram of FIG. 13). While daily delivery of TBZ or VBZ showed a large plasma fluctuation of the VMAT2 inhibitors, all (+)-(α)-DHTBZ-polymer solution formulations enabled a substantially smaller P/T ratio of VMAT2 inhibitors. Notably, consistent with what was found in the in vitro studies, (+)-(α)-DHTBZ particle size indeed showed no effect on the in vivo rat PK profile. This finding was advantageous for that API particle size range might not need to be set in a narrow range, which would be beneficial from product development point of view. All and all, the (+)-(α)-DHTBZ formulations presented in this application undoubtedly posed great potential to be used as sustained release medications requiring much lower dosing frequency, yet are able to continuously deliver VMAT2 inhibitors within effective therapeutic ranges, which shall significantly reduce side-effects accompany with currently available treatment of XENAZINE®.

### Example 12. PK of Subcutaneously Administrated (+)-(α)-DHTBZ-PLGA 5050/NMP Formulations in Rats

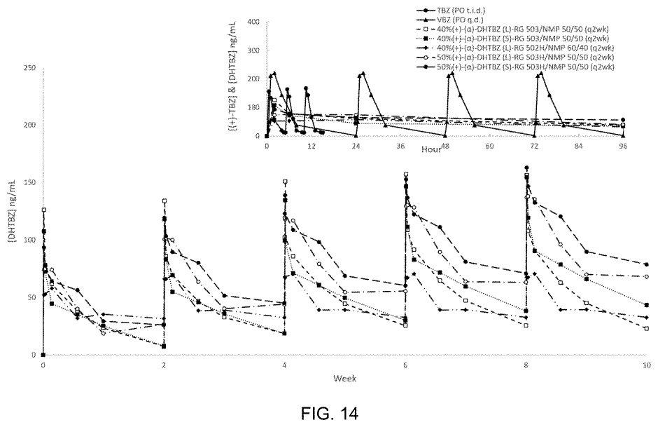

In vivo animal studies were conducted with SD rats using formulations composed of: 40% (+)-(α)-DHTBZ (L)-RG 503/NMP at 50/50 w/w ratio, 40% (+)-(α)-DHTBZ (S)-RG 503/NMP at 50/50 w/w ratio, 40% (+)-(α)-DHTBZ (L)-RG 502H/NMP at 60/40 w/w ratio, 50% (+)-(α)-DHTBZ (L)-RG 503H/NMP at w/w 50/50 ratio, and 50% (+)-(α)-DHTBZ (S)-RG 503H/NMP at 50/50 w/w ratio. All suspensions were selected for their sustained in vitro release without high initial burst as demonstrated in the earlier examples in this application. (+)-(α)-DHTBZ polymer depot compositions were prepared as previously described. All five formulations described above were subcutaneously administrated to SD rats (N=3) at a dose level of 50 mg/kg, while others received TBZ or VBZ suspensions at a dose level of 10 mg/kg via oral gavage (N=3) as references. The animals received (+)-(α)-DHTBZ-polymer suspensions were dosed on Day 1, followed by blood sampling at 2, 6, 12, 24 hours and 4, 7, 14, 21, 28, 35, 42, 49, 56 days post-dosing. For the animals that received TBZ or VBZ suspensions orally, blood sampling was performed at 2, 6, 12, 24, and 48 hours post-dosing. For each animal, plasma (+)-(α)-DHTBZ concentrations were measured via LC-MS to evaluate the 35-Day PK profiles. Furthermore, PK simulation for bi-weekly repeated dosing of the (+)-(α)-DHTBZ-RESOMER suspensions as well as the daily repeated dosing of TBZ or VBZ were obtained and presented as FIG. 14. The lower large diagram of FIG. 14 demonstrated that all (+)-(α)-DHTBZ-polymer suspensions were able to successfully provide low P/T ratios, yet sustained release of VMAT2 inhibitors over 2 weeks. In addition, throughout the PK study, plasma levels of VMAT2 inhibitors released from the polymeric formulations fall in between a window of 10-200 ng/mL, which is in the range of the plasma level given by the oral dosage form of VBZ. More significantly, the present application again, enabled the polymer depot compositions which can provide sustained release of VMAT2 inhibitors with significantly less plasma level fluctuation, comparing to other commercially available drug products. PK simulation of thrice-a-day repeated dosing of TBZ and once-a-day repeated dosing of VBZ were compared side-by-side with PK simulations of bi-weekly repeated dosing of formulations composed of 40% (+)-(α)-DHTBZ (L)-RG 503/NMP at 50/50 w/w ratio, 40% (+)-(α)-DHTBZ (S)-RG 503/NMP at 50/50 w/w ratio, 40% (+)-(α)-DHTBZ (L)-RG 502H/NMP at 60/40 w/w ratio, 50% (+)-(α)-DHTBZ (L)-RG 503H/NMP at w/w 50/50 ratio, and 50% (+)-(α)-DHTBZ (S)-RG 503H/NMP at 50/50 w/w ratio (Top, small diagram of FIG. 14). While daily delivery of TBZ or VBZ showed a large plasma fluctuation of the VMAT2 inhibitors, all (+)-(α)-DHTBZ-polymer solution formulations enabled significantly reduced P/T ratio of VMAT2 inhibitors. Notably, the (+)-(α)-DHTBZ formulations presented in this application undoubtedly posed great potential to be used as sustained release medications requiring much lower dosing frequency, yet are able to continuously deliver VMAT2 inhibitors within effective therapeutic ranges, which shall significantly reduce side-effects accompany with currently available treatment of XENAZINE®.

What is claimed is:

1. An injectable polymer depot composition comprising: a) a VMAT2 inhibitor, a deuterated derivative thereof, a pharmaceutically acceptable salt thereof, an active metabolite thereof, or a prodrug thereof; b) a biodegradable polymer selected from the group consisting of a homopolymer polylactide or polylatic acid (PLA), a copolymer poly (lactic acid-co-glycolic acid) or poly (lactide-co-glycolide) (PLGA), and a combination thereof, wherein the PLGA has a monomer ratio of lactide:glycolide (or lactic acid:glycolic acid) between 50:50 and 99:1 inclusive; and c) a pharmaceutically acceptable organic solvent selected from the group consisting of N-methyl-2-pyrrolidone, 2-pyrrolidone, N,N-dimethylformamide, dimethyl sulfoxide, benzyl alcohol, benzyl benzoate, and combinations thereof.

2. The injectable polymer depot composition of claim 1, wherein the VMAT2 inhibitor is (+)-TBZ.

3. The injectable polymer depot composition of claim 1, wherein the VMAT2 inhibitor is (+)-(α)-DHTBZ.

4. The injectable polymer depot composition of claim 1, wherein the pharmaceutically acceptable organic solvent is N-methyl-2-pyrrolidone.

5. The injectable polymer depot composition of claim 1, wherein the amount of VMAT2 inhibitor ranges from 5 to 70% by weight in the polymer depot composition.

6. The injectable polymer depot composition of claim 1 comprising 10% to 90% by weight of the biodegradable polymer.

7. The injectable polymer depot composition of claim 1, wherein the biodegradable polymer has a monomer ratio of lactic acid to glycolic acid of about 100:0, 95:5, 90:10, 85:15, 80:20, 75:25, 70:30, 65:35, 60:40, 55:45, or 50:50.

8. The injectable polymer depot composition of claim 1, wherein the biodegradable polymer comprises a group selected from the group consisting of ester terminal functional groups, carboxylic acid terminal functional groups, hydroxyl groups, and combinations thereof.

9. The injectable polymer depot composition of claim 1, wherein the biodegradable polymer has an average weight molecular weight (Mw) of between 5,000 to 120,000.

10. The injectable polymer depot composition of claim 1 comprising 10% to 90% by weight of the pharmaceutically acceptable organic solvent based on the total amount of the biodegradable polymer and the pharmaceutically acceptable organic solvent.

11. The injectable polymer depot composition of claim 1 further comprising an additive selected from the group consisting of stabilizing agents, antioxidants, buffering agents, release modifying agents, and combinations thereof.

12. The injectable polymer depot composition of claim 1, wherein the VMAT2 inhibitor has a particle size distribution characterized by D(50) in a range from about 2 μm to about 300 μm.

13. The injectable polymer depot composition of claim 1, wherein the VAMT2 inhibitor is uniformly dispersed in the composition and is filled in a syringe for subcutaneous or intramuscular injection.

14. A method of making the injectable polymer depot composition of claim 1, comprising: prefilling the VAMT2 inhibitor, the deuterated derivative, the salt, the active metabolite, or the prodrug thereof in a syringe A; homogeneously mixing the biodegradable polymer and the pharmaceutically acceptable organic solvent to form a homogeneous mixture, and prefilling the homogeneous mixture in a syringe B; thoroughly mixing components in the syringes A and B to form the polymer depot composition.

15. A method of treating hyperkinetic movement disorder comprising administering a patient in need thereof the composition of claim 1 via injection; forming an in situ sustained release implant/depot upon administering to the patient; gradually releasing the VMAT2 inhibitor, the deuterated derivative thereof, the pharmaceutically acceptable salt thereof, the active metabolite thereof, or the prodrug thereof to the patient for at least one week.

16. The method of claim 15 comprising, after the administering, providing sustained release of the VMAT2 inhibitor, the deuterated derivative thereof, the pharmaceutically acceptable salt thereof, the active metabolite thereof, or the prodrug thereof with a plasma level peak/trough (P/T) ratio between 1 to 10.

17. The method of claim 15, wherein release of the VMAT2 inhibitor, the deuterated derivative thereof, the pharmaceutically acceptable salt thereof, the active metabolite thereof, or the prodrug thereof from the in situ sustained release implant/depot is no more than 30% of total VMAT2 inhibitor loading 24 hours after the administration.

18. The method of claim 15 wherein the hyperkinetic movement disorder is selected from the group consisting of tardive dyskinesia (TD), chorea associated with Huntington's disease (HD), tremors, dystonia, tics, myoclonus, stereotypies, restless legs syndrome, and various other disorders with abnormal involuntary movements.
